SEQUENCE LISTING (46 sequences)

SEQ ID NO: 1 - protein, 6 aa, Artificial Sequence
  Synthetic peptide
  MISC_FEATURE (1)..(1): D-amino acid
  MISC_FEATURE (1)..(1): N-methylated
  MISC_FEATURE (5)..(5): N-methylated
RGDAAA

SEQ ID NO: 2 - protein, 6 aa, Artificial Sequence
  Synthetic peptide
  MISC_FEATURE (1)..(1): D-amino acid
  MISC_FEATURE (1)..(1): N methylated
  MISC_FEATURE (6)..(6): N methylated
ARGDAA

SEQ ID NO: 3 - protein, 6 aa, Artificial Sequence
  Synthetic peptide
  MISC_FEATURE (1)..(1): D-amino acid
  MISC_FEATURE (3)..(3): N methylated
  MISC_FEATURE (5)..(5): N methylated
RGDAAA

SEQ ID NO: 4 - protein, 6 aa, Artificial Sequence
  Synthetic peptide
  MISC_FEATURE (1)..(1): D-amino acid
  MISC_FEATURE (5)..(5): N methylated
  MISC_FEATURE (6)..(6): N methylated
RGDAAA

SEQ ID NO: 5 - protein, 6 aa, Artificial Sequence
  Synthetic peptide
  MISC_FEATURE (1)..(1): D-amino acid
  MISC_FEATURE (1)..(1): N methylated
  MISC_FEATURE (6)..(6): N methylated
VRGDAA

SEQ ID NO: 6 - protein, 6 aa, Artificial Sequence
  Synthetic peptide
  MISC_FEATURE (1)..(1): D-amino acid
  MISC_FEATURE (1)..(1): N methylated
  MISC_FEATURE (6)..(6): N methylated
FRGDAA

SEQ ID NO: 7 - protein, 6 aa, Artificial Sequence
  Synthetic peptide
  MISC_FEATURE (1)..(1): D-amino acid
  MISC_FEATURE (1)..(1): N methylated
  MISC_FEATURE (5)..(5): N methylated
RGDAAV

SEQ ID NO: 8 - protein, 6 aa, Artificial Sequence
  Synthetic peptide
  MISC_FEATURE (1)..(1): D-amino acid
  MISC_FEATURE (1)..(1): N methylated
  MISC_FEATURE (5)..(5): N methylated
RGDAAF

SEQ ID NO: 9 - protein, 6 aa, Artificial Sequence
  Cyclic peptide
  MISC_FEATURE (1)..(1): D-amino acid
  MISC_FEATURE (1)..(1): N methylated
  MISC_FEATURE (2)..(2): Two hexyloxycarbonyl (Hoc) moieties
  MISC_FEATURE (4)..(4): Methyl ester (OMe) moiety
  MISC_FEATURE (6)..(6): N methylated
VRGDAA

SEQ ID NO: 10 - protein, 6 aa, Artificial Sequence
  Cyclic peptide
  MISC_FEATURE (1)..(1): D-amino acid
  MISC_FEATURE (1)..(1): N methylated
  MISC_FEATURE (2)..(2): Two hexyloxycarbonyl (Hoc) moieties
  MISC_FEATURE (4)..(4): Methyl ester (OMe) moiety
  MISC_FEATURE (6)..(6): N methylated
ARGDAA

SEQ ID NO: 11 - protein, 6 aa, Artificial Sequence
  Cyclic peptide
  MISC_FEATURE (1)..(1): D-amino acid
  MISC_FEATURE (1)..(1): N methylated
  MISC_FEATURE (1)..(1): Two hexyloxycarbonyl (Hoc) moieties
  MISC_FEATURE (3)..(3): Methyl ester (OMe) moiety
  MISC_FEATURE (5)..(5): N methylated
RGDAAA

SEQ ID NO: 12 - protein, 6 aa, Artificial Sequence
  Cyclic peptide
  MISC_FEATURE (1)..(1): D-amino acid
  MISC_FEATURE (1)..(1): Two hexyloxycarbonyl (Hoc) moieties
  MISC_FEATURE (3)..(3): Methyl ester (OMe) moiety
  MISC_FEATURE (5)..(5): N methylated
  MISC_FEATURE (6)..(6): N methylated
RGDAAA

SEQ ID NO: 13 - protein, 6 aa, Artificial Sequence
  Synthetic peptide
  MISC_FEATURE (1)..(1): D-amino acid
  MISC_FEATURE (1)..(1): N methylated
  MISC_FEATURE (6)..(6): N methylated
RGDAAA

SEQ ID NO: 14 - protein, 6 aa, Artificial Sequence
  Synthetic peptide
  MISC_FEATURE (1)..(1): D-amino acid
  MISC_FEATURE (1)..(1): N methylated
  MISC_FEATURE (2)..(2): Two hexyloxycarbonyl (Hoc) moieties
  MISC_FEATURE (4)..(4): Methyl ester (OMe) moiety
  MISC_FEATURE (6)..(6): N methylated
FRGDAA

SEQ ID NO: 15 - protein, 9 aa, Artificial Sequence
  Synthetic peptide
  MISC_FEATURE (5)..(5): Two hexyloxycarbonyl (Hoc) moieties
LPPFRGDLA

SEQ ID NO: 16 - protein, 8 aa, Artificial Sequence
  Synthetic peptide
  MISC_FEATURE (6)..(6): Two hexyloxycarbonyl (Hoc) moieties
LPPGLRGD

SEQ ID NO: 17 - protein, 9 aa, Artificial Sequence
  Synthetic peptide
LPPFRGDLA

SEQ ID NO: 18 - protein, 8 aa, Artificial Sequence
  Synthetic peptide
LPPGLRGD

SEQ ID NO: 19 - protein, 6 aa, Artificial Sequence
  Synthetic peptide
  MISC_FEATURE (1)..(1): D-amino acid
AAAAAA

SEQ ID NO: 20 - protein, 5 aa, Artificial Sequence
  Synthetic peptide
  MISC_FEATURE (1)..(1): D-amino acid
  MISC_FEATURE (2)..(2): N methylated
FVRGD

SEQ ID NO: 21 - protein, 6 aa, Artificial Sequence
  Synthetic peptide
  MISC_FEATURE (1)..(1): D-amino acid
  MISC_FEATURE (1)..(1): N methylated
  MISC_FEATURE (2)..(2): Hexyloxycarbonyl (Hoc) moiety
  MISC_FEATURE (4)..(4): Methyl ester (OMe) moiety
  MISC_FEATURE (6)..(6): N methylated
VRGDAA

SEQ ID NO: 22 - protein, 6 aa, Artificial sequence
  Synthetic peptide
  MISC_FEATURE (1)..(1): D-amino acid
  MISC_FEATURE (2)..(2): N methylayed
  MISC_FEATURE (3)..(3): Two hexyloxycarbonyl (Hoc) moieties
AAAAAA

SEQ ID NO: 23 - protein, 5 aa, Artificial sequence
  Synthetic peptide
  MISC_FEATURE (1)..(1): D-amino acid
  MISC_FEATURE (2)..(2): N methylated
  MISC_FEATURE (3)..(3): Two hexyloxycarbonyl (Hoc) moieties
FVRGD

SEQ ID NO: 24 - protein, 6 aa, Artificial Sequence
  Cyclic peptide
  MISC_FEATURE (1)..(1): D-amino acid
  MISC_FEATURE (1)..(1): N-methylated
  MISC_FEATURE (5)..(5): N-methylated
RGDAAA

SEQ ID NO: 25 - protein, 6 aa, Artificial Sequence
  Cyclic peptide
  MISC_FEATURE (1)..(1): D-amino acid
  MISC_FEATURE (1)..(1): N methylated
  MISC_FEATURE (6)..(6): N methylated
ARGDAA

SEQ ID NO: 26 - protein, 6 aa, Artificial Sequence
  Cyclic peptide
  MISC_FEATURE (1)..(1): D-amino acid
  MISC_FEATURE (3)..(3): N methylated
  MISC_FEATURE (5)..(5): N methylated
RGDAAA

SEQ ID NO: 27 - protein, 6 aa, Artificial Sequence
  Cyclic peptide
  MISC_FEATURE (1)..(1): D-amino acid
  MISC_FEATURE (5)..(5): N methylated
  MISC_FEATURE (6)..(6): N methylated
RGDAAA

SEQ ID NO: 28 - protein, 6 aa, Artificial Sequence
  Cyclic peptide
  MISC_FEATURE (1)..(1): D-amino acid
  MISC_FEATURE (1)..(1): N methylated
  MISC_FEATURE (6)..(6): N methylated
VRGDAA

SEQ ID NO: 29 - protein, 6 aa, Artificial Sequence
  Cyclic peptide
  MISC_FEATURE (1)..(1): D-amino acid
  MISC_FEATURE (1)..(1): N methylated
  MISC_FEATURE (6)..(6): N methylated
FRGDAA

SEQ ID NO: 30 - protein, 6 aa, Artificial Sequence
  Cyclic peptide
  MISC_FEATURE (1)..(1): D-amino acid
  MISC_FEATURE (1)..(1): N methylated
  MISC_FEATURE (5)..(5): N methylated
RGDAAV

SEQ ID NO: 31 - protein, 6 aa, Artificial Sequence
  Cyclic peptide
  MISC_FEATURE (1)..(1): D-amino acid
  MISC_FEATURE (1)..(1): N methylated
  MISC_FEATURE (5)..(5): N methylated
RGDAAF

SEQ ID NO: 32 - protein, 6 aa, Artificial Sequence
  Synthetic peptide
  MISC_FEATURE (1)..(1): D-amino acid
  MISC_FEATURE (1)..(1): N methylated
  MISC_FEATURE (2)..(2): Two hexyloxycarbonyl (Hoc) moieties
  MISC_FEATURE (4)..(4): Methyl ester (OMe) moiety
  MISC_FEATURE (6)..(6): N methylated
VRGDAA

SEQ ID NO: 33 - protein, 6 aa, Artificial Sequence
  Synthetic peptide
  MISC_FEATURE (1)..(1): D-amino acid
  MISC_FEATURE (1)..(1): N methylated
  MISC_FEATURE (2)..(2): Two hexyloxycarbonyl (Hoc) moieties
  MISC_FEATURE (4)..(4): Methyl ester (OMe) moiety
  MISC_FEATURE (6)..(6): N methylated
ARGDAA

SEQ ID NO: 34 - protein, 6 aa, Artificial Sequence
  Synthetic peptide
  MISC_FEATURE (1)..(1): D-amino acid
  MISC_FEATURE (1)..(1): N methylated
  MISC_FEATURE (1)..(1): Two hexyloxycarbonyl (Hoc) moieties
  MISC_FEATURE (3)..(3): Methyl ester (OMe) moiety
  MISC_FEATURE (5)..(5): N methylated
RGDAAA

SEQ ID NO: 35 - protein, 6 aa, Artificial Sequence
  Synthetic peptide
  MISC_FEATURE (1)..(1): D-amino acid
  MISC_FEATURE (1)..(1): Two hexyloxycarbonyl (Hoc) moieties
  MISC_FEATURE (3)..(3): Methyl ester (OMe) moiety
  MISC_FEATURE (5)..(5): N methylated
  MISC_FEATURE (6)..(6): N methylated
RGDAAA

SEQ ID NO: 36 - protein, 6 aa, Artificial Sequence
  Cyclic peptide
  MISC_FEATURE (1)..(1): D-amino acid
  MISC_FEATURE (1)..(1): N methylated
  MISC_FEATURE (6)..(6): N methylated
RGDAAA

SEQ ID NO: 37 - protein, 6 aa, Artificial Sequence
  Cyclic peptide
  MISC_FEATURE (1)..(1): D-amino acid
  MISC_FEATURE (1)..(1): N methylated
  MISC_FEATURE (2)..(2): Two hexyloxycarbonyl (Hoc) moieties
  MISC_FEATURE (4)..(4): Methyl ester (OMe) moiety
  MISC_FEATURE (6)..(6): N methylated
FRGDAA

SEQ ID NO: 38 - protein, 9 aa, Artificial Sequence
  Cyclic peptide
  MISC_FEATURE (5)..(5): Two hexyloxycarbonyl (Hoc) moieties
LPPFRGDLA

SEQ ID NO: 39 - protein, 8 aa, Artificial Sequence
  Cyclic peptide
  MISC_FEATURE (6)..(6): Two hexyloxycarbonyl (Hoc) moieties
LPPGLRGD

SEQ ID NO: 40 - protein, 9 aa, Artificial Sequence
  Cyclic peptide
LPPFRGDLA

SEQ ID NO: 41 - protein, 8 aa, Artificial Sequence
  Cyclic peptide
LPPGLRGD

SEQ ID NO: 42 - protein, 6 aa, Artificial Sequence
  Cyclic peptide
  MISC_FEATURE (1)..(1): D-amino acid
AAAAAA

SEQ ID NO: 43 - protein, 5 aa, Artificial Sequence
  Cyclic peptide
  MISC_FEATURE (1)..(1): D-amino acid
  MISC_FEATURE (2)..(2): N methylated
FVRGD

SEQ ID NO: 44 - protein, 6 aa, Artificial Sequence
  Cyclic peptide
  MISC_FEATURE (1)..(1): D-amino acid
  MISC_FEATURE (1)..(1): N methylated
  MISC_FEATURE (2)..(2): Hexyloxycarbonyl (Hoc) moiety
  MISC_FEATURE (4)..(4): Methyl ester (OMe) moiety
  MISC_FEATURE (6)..(6): N methylated
VRGDAA

SEQ ID NO: 45 - protein, 6 aa, Artificial sequence
  Cyclic peptide
  MISC_FEATURE (1)..(1): D-amino acid
  MISC_FEATURE (2)..(2): N methylayed
  MISC_FEATURE (3)..(3): Two hexyloxycarbonyl (Hoc) moieties
AAAAAA

SEQ ID NO: 46 - protein, 5 aa, Artificial sequence
  Cyclic peptide
  MISC_FEATURE (1)..(1): D-amino acid
  MISC_FEATURE (2)..(2): N methylated
  MISC_FEATURE (3)..(3): Two hexyloxycarbonyl (Hoc) moieties
FVRGD

(19) United States
(12) Patent Application Publication
(10) Pub. No.: US 2022/0288156 A1
(43) Pub. Date: Sep. 15, 2022

(54) TREATMENT

(71) Applicant: QUEEN MARY UNIVERSITY OF LONDON, London (GB)

(72) Inventor: Kairbaan HODIVALA-DILKE, London Greater London (GB)

(21) Appl. No.: 17/635,595

(22) PCT Filed: Aug. 14, 2020
(86) PCT No.: PCT/GB2020/051944

§ 371 Date: Feb. 15, 2022

(30) Foreign Application Priority Data
Aug. 16, 2019 (GB) 1911816.5

(51) Int. Cl. A61K 38/12; A61K 38/07; A61P 9/04; A61P 9/10

(52) U.S. Cl. CPC A61K 38/12; A61K 38/07; A61P 9/04; A61P 9/10

(57) ABSTRACT

The invention relates to the prevention or treatment of a vascular condition or heart failure. It relates to the medical use of an αvβ3- and/or αvβ5-integrin targeting agent for such a purpose.

### FIELD OF THE INVENTION

The invention relates to the prevention or treatment of a vascular condition or heart failure. It relates to the medical use of an αvβ3- and/or αvβ5-integrin targeting agent for such a purpose.

### BACKGROUND TO THE INVENTION

The provision of new therapies for vascular conditions and heart failure is of high importance. Heart failure affects approximately 26 million people worldwide and is characterised by insufficient cardiac function, poor coronary microvascular reactivity and maladaptive cardiomyocyte hypertrophy. Despite significant therapeutic advances, none of the current treatment strategies provide long-term benefit since the disease is associated with a 5-year mortality of almost 50% (1-11). Given also that current standard-of-care treatments, such as β-blockers or diuretics, are generally administered indefinitely, can cause serious side effects and do not fully mitigate heart failure-related morbidity and mortality, alternative, disease modulating solutions are still required.

Integrins are heterodimeric transmembrane adhesion molecules composed of one a-subunit and one β-subunit. Heterodimer composition confirms ligand specificity and integrin-heterodimer profiles are cell type specific and influenced by environment, age and oxygen availability (12). Apart from their roles in adhesion, integrins have also been shown to be allosteric thus affecting their signalling capacity. Signalling downstream of integrins can alter the activity of other cell surface receptors including growth factor receptors (12). The clinical application of inhibiting integrin adhesive functions in vivo has proved to be effective (i.e. the anti-platelet effects of αIIβ3 integrin antagonists), but targeting the vitronectin receptor, αvβ3-integrin (13, 14), appears to be less successful. Cilengitide was developed originally as an anti-angiogenic/anti-cancer cyclic RGD-mimetic antagonist of avb3 integrin when used at maximally tolerated doses (5-50 mg/kg) (22-26). In contrast to its antagonistic anti-adhesive and anti-angiogenic effects at these doses, low doses of Cilengitide (ldCil, 50 μg/kg in vivo or 2 nM in vitro) enhance pathological angiogenesis with no apparent effects on quiescent vasculature. These features of ldCil were exploited to improve chemotherapy efficacy in cancer models in vivo by actually increasing tumour blood vessel number and directly enhancing chemotherapy delivery and metabolism in malignant cells (28).

### SUMMARY OF THE INVENTION

The inventors have surprisingly shown that an αvβ3- and/or αvβ5-integrin targeting agent, as illustrated by cilengitide, is able to promote normal angiogenesis, endothelial cell activation and cardiomyocyte function, outside of the context of pathological tumour angiogenesis. Cilengitide is able to restore normal function in a mouse model of heart failure, promoting integrin signalling conducive to cardiac angiogenesis and restoring cardiomyocyte function, and inhibiting hypertrophy. A long lasting duration of effect after treatment was also observed. The effects illustrated in the model of heart failure are also expected to be useful in therapies for other conditions involving malfunctioning vasculature where αvβ3- and/or αvβ5 integrins can be targeted similarly to activate endothelial cells and promote normal angiogenesis and vascular function.

αvβ3- and/or αvβ5-integrin targeting agents may thus be used advantageously in prevention and treatment of a vascular condition or heart failure and may be beneficially provided in combination with other agents suitable for preventing or treating a vascular condition, heart failure or symptoms of heart failure.

The invention thus provides a method of preventing or treating a vascular condition or heart failure in a patient, comprising administering an αvβ3- and/or αvβ5-integrin targeting agent to the patient.

The invention also provides an αvβ3- and/or αvβ5-integrin targeting agent for use in a method of preventing or treating a vascular condition or heart failure in a patient.

The invention further provides a combination of an αvβ3- and/or αvβ5-integrin targeting agent and at least one additional agent suitable for preventing or treating a vascular condition, heart failure or symptoms of heart failure.

### BRIEF DESCRIPTION OF THE FIGURES

FIG. 1 shows β3-integrin expression is upregulated in human dilated and ischaemic cardiomyopathy. (A) Western blot analysis of β3-integrin in protein lysates from human non-failing, non-failing with hypertrophy, dilated and ischaemic human myocardium. GAPDH acts as the loading control. Bar charts represent mean densitometric readings±SEM n, 5-6 separate patient samples. (B) Immunofluorescence reveals β3-integrin expression in sections of human non-failing heart with an apparent increase in dilated and ischaemic human hearts but less so in non-failing with hypertrophic heart. Non-specific primary antibody control shows minimal background level staining. N, 4 separate patient samples per group. Scale bar, 50 μm. Students t-test, *p<0.05.

FIG. 2 shows that low dose Cilengitide treatment restores the effect of pressure overload induced by abdominal aortic constriction. (A) Wild type mice underwent either SHAM, or abdominal aortic constriction (AAC) surgery and were either treated with Cilengitide (Cil, 50 μg/kg or 500 μg/kg) or vehicle alone immediately after surgery in a preventative mode trial. Representative endpoint echocardiographic images in vehicle and Cil treated SHAM-surgery or AAC-surgery mice (n, 7 mice per group). Double-headed arrows, LV internal diameter at diastole (LVIDd) and systole (LVIDs). (B) Treatment of mice with 50 μg/kg, but not 500 μg/kg, Cilengitide corrects the fractional shortening effects after AAC surgery. Line graphs represent summary fractional shortening (FS %) analysis over time and bar chart, end point FS % from the mice treated in (A). (C) Three weeks after AAC surgery both FS % and ejection fraction (EF %) are reduced, whilst left ventricular internal diameter (during systole) (LVID;s) was enhanced compared with SHAM controls (n, 5 mice per group). (D) Treatment of mice with ldCil rescues the fractional shortening effects of AAC surgery in an intervention mode trial. Wild type mice underwent either SHAM or AAC surgery and 3 weeks after surgery were treated with either vehicle alone or 50 μg/kg Cil (low dose Cilengitide, ldCil) 3 times a week for 3 weeks. Representative endpoint echocardiographic images in SHAM-surgery or AAC-surgery mice treated with either vehicle or ldCil from 3-6 weeks post-surgery (n, 7 mice per group). Line graphs, summary fractional shortening (FS %) over time. (E) Functional parameters from mice in (D): Endpoint FS %, EF % and LVID;s were rescued after ldCil treatment in mice after AAC surgery. Mean arterial blood pressure (MABP) was not affected by ldCil treatment (n, 3 mice per group). (F) Sections of myocardium from mice in (D) stained with wheat germ agglutinin to detect cell perimeters. Image analysis of cardiomyocyte perimeter indicates that treatment with ldCil is sufficient to rescue the myocyte size defect compared with mice with SHAM surgery. (n, 3 mice per group). (G) Quantitation of heart weight normalised to body weight across treatment groups. (n, 7 mice per group). (H) Treatment with ldCil provides sustained recovery after AAC surgery. Wild type mice underwent either SHAM or AAC surgery. From 3-6 weeks post-surgery mice were either treated with ldCil, or vehicle alone as control and fractional shortening then measured at 0, 3, 6, and 12 weeks post-surgery. Line graphs, summary FS % over time. Results show that at 3 weeks post-AAC surgery FS % is significantly reduced in mice after AAC surgery and that this effect did not recover in mice given vehicle alone. In contrast, mice treated with ldCil from 3-6 weeks post AAC surgery showed recovery in FS % that was sustained for 6 weeks after treatment cessation (n, 3-6 mice per group). Data are shown as mean±SEM. Statistical analysis (B-G) one-way ANOVA with Tukey's post hoc analysis. Statistical analysis in (H) non-parametric test Mann-Whitney. *p<0.05, **p<0.01. NS, not significant. Scale bar in (F) 20 μm.

FIG. 3 shows that treatment with low dose Cilengitide enhances cardiac blood vessel density in vivo and stimulates cardiac endothelial cell DNA-replication, mitosis and cell-cycle transition transcriptional profiles. (A) Wild type mice underwent either SHAM or AAC surgery and were treated with ldCil or vehicle control for 3 weeks. Representative images of myocardium immunostained for the endothelial cell marker CD31. Quantitation shows that treatment with ldCil enhances blood vessel density (BVD) in the myocardium of mice after AAC but not SHAM surgery. Data are given as mean±SEM. Statistical analysis by one-way ANOVA with Tukey's post hoc analysis; *p<0.05. NS, not significant. (B) Duplicate preparations of mouse cardiac endothelial cells were treated for 0, 24 or 48 h with ldCil and RNA-Seq performed. Heatmap of differentially expressed (DE) up- and down-regulated transcripts in cardiac endothelial cells treated with ldCil. Results are shown in duplicate. 54 DE genes were concordant at 24 hr and 48 hr ldCil treatment compared with control. (C) Enrichment analysis of RNA-Seq data demonstrates that treatment of cardiac endothelial cells significantly enriches for pathways in cell cycle, DNA replication, cell cycle, mitosis, and DNA strand elongation all pathways involved in cell proliferation a key process in angiogenesis.

FIG. 4 shows that low dose Cilengitide treatment reverses the transcriptomic profiles of AngII-stimulated cardiomyocytes to control levels. (A) Mouse cardiomyocytes were isolated and either exposed to angiotensin II (AngII) to mimic the molecular stress of heart failure or exposed to AngII and also treated with ldCil. Heat map of differentially expressed protein coding genes indicates that AngII-stimulation induces 14 down- and 9 up-regulated transcripts. ldCil treatment of AngII-stimulated cardiomyocytes rescues the differentially expressed transcript profiles. (B) Data from (A) represented in fold change order. DE protein-coding genes with **p<0.01 in AngII vs control, also with high average expression across samples, became not DE (p>0.05) in AngII+ldCil group compared to control. Green bars (third set of bars from the left) show levels of fold change. (C) Treatment with ldCil restores aberrant signalling pathways induced by AngII-stimulation of cardiomyocytes. GSEA of KEGG and reactome signalling pathways reveal that dysregulated pathways with FDR q<0.1 in AngII vs control, became not significant (q>0.1) in AngII+ldCil compared to control.

FIG. 5 shows that low dose Cilengitiide restores transcriptomic profiles similar to that found in non-failing human heart. (A) Treatment of mouse cardiomyocytes with AngII+ldCil compared with AngII restores transcriptomic signature similar to that found in human non-failing vs failing heart. Heatmap illustrates concordant DE transcripts between human failing (ischaemic and dilated cardiomyopathies) vs non-failing heart and mouse cardiomyocytes treated with AngII vs Control or AngII vs AngII+ldCil. (B) Venn diagrams show the overlap of gene sequence expression analysis (GSEA) pathways (p<0.05) in mouse cardiomyocytes stimulated with AngII vs control and human dilated CMP vs non-failing heart. Scatterplots of Normalised Enrichment Scores (NES) of the overlapping pathways illustrate how many of those change concordantly. Heatmap of NES for GSEA pathways that change concordantly in human dilated CMP vs non-failing heart, mouse AngII-stimulated vs control cardiomyocytes and AngII-stimulated cardiomyocytes treated with ldCil vs AngII-stimulated cardiomyocytes. (C) Venn diagrams of the overlap of GSEA pathways (p<0.05) in mouse cardiomyocytes stimulated with AngII vs control and human ischemic CMP vs non-failing heart. Scatterplots of NES of the overlapping pathways illustrate how many of those change concordantly. Heatmap of NES for GSEA pathways that change concordantly in human ischemic vs non-failing heart mouse AngII-stimulated vs control cardiomyocytes, AngII-stimulated cardiomyocytes treated with ldcil vs AngII-stimulated cardiomyocytes.

FIG. 6 shows the histological changes in human non-failing, non-failing hypertrophic, ischaemic and dilated cardiomyopathies. Representative images of Haemotoxylin & Eosin (H&E) and Masson's trichrome stained sections of non-failing, non-failing hearts with hypertrophy, ischaemic and dilated cardiomyopathy. Non-failing with hypertrophy heart shows an increase in myocyte size. Ischaemic and dilated cardiopathies show elevated fibrotic responses. Black arrows, fibrosis. Scale bars, (A) 500 μm; (B) 100 μm.

FIG. 7 shows that AAC surgery does not affect cardiac blood vessel density. Wild type mice underwent either SHAM or AAC surgery. (A) Bar chart represents numbers of CD31-positive blood vessels/high power field of view (40×) of hearts from mice that underwent SHAM surgery or 2, 3, and 6 weeks post AAC surgery. (B) Representative images of CD31-immunostaining in mouse myocardium. Data are shown as mean±SEM. Statistical analysis by 1-way ANOVA with Tukey's multiple comparison post hoc, NS is not significant. Scale bar, 20 μm.

FIG. 8 shows transcriptomic changes in cardiac endothelial cells after treatment with low dose Cilengitide. Cardiac endothelial cells were isolated and treated with vehicle alone (control) for 24 h or 48 h with low dose Cilengitide (ldCil, 20 nM). Two separate samples were prepared for each condition. (A) At 24 h of 20 nM Cilengitide treatment 146 genes were DE (FDR q<0.05 and absolute log 2FC>1). Genes that are upregulated after IdCil treatment are: Btbd11, Dyncli1, Gm38287, St8sia1, Htr2a, Gdf15, Apln, Adam12, Olfml3, Galnt17, Uchl4, Ube2ql1, Opcml, Gm43102, Crispld2, ligp1, Eln, Csf2rb, Csf2rb2, Ccl7, Nov, Mt2, Socs2, Plet1, Gm14221, Mycl, Slc30a1, Col6a3, Mt1, Ggt5, Adam8, Fgf23, Gbp4, Eme1, AA467197 and Pbp2. Genes that are downregulated after IdCil treatment are: Ehd3, Aldh3a1, Hey1, Gm26885, Hey2, Cxcl1, Col4a4, Cyp24a1, Gm36283, Mal, Lmntd2, Gm31024, Galnt15, Slc10a6, Ahsg, Clec4d, Prelp, Tmem132c, Cyp2d22, Epor, Apoe, Clec14a, Dkk3, C1ql4, Slc2a13, Lrg, A130014A01Rik, Plekha6, Rubcnl, 2310014F06Rik, Ephx2, Cd300lg, Mgat3, Tmc3, Gm16386, Mir351, Cytl1, Frat1, 2900041M22Rik, Brinp2, Colca2, Gm11944, 2510016D11Rik, Lama3, Mylip, Tcp11l2, Fam84b, Nr4a2, Slc25a34, Rspo3, Gm31166, 2410022M11Rik, Ebf4, C8g, Kif5c, Shisa2, C1qtnf7, Pdgfrb, Smoc1, Pcsk5, Dock10, Bcl6, Gpr160, Cdkn1c, Oprl1, Insc, Aldoc, Pdlim3, Pygl, Crispld1, 9930014A18Rik, Celf4, Npr3, Pdk4, Ccdc157, Tbx20, Chrm2, Gstm7, Cdo1, Sox9, Casp6, Ankrd55, Wnk4, Ank, Cfi, Klf15, Aldh1a7, Gnao1, Col4a3, Colec11, Adamtsl2, Enpp1, Fgfr2, Ptprq, Lsamp, Mme, Pdgfra, Trabd2b, Tnfrsf11b, Emb, Mpl, Scd2, Scd1, Gpc3, Scel, Smad9, Gm28438, Syne4, Comp, and Aass. (B) At 48 h of 20 nM Cilengitide treatment 107 genes were DE (FDR q<0.05 and absolute log 2FC>1). Genes that are upregulated after IdCil treatment are: Thbs2, Enpp2, Trp53i11, Fgl2, Tnfrsf25, Il2rg, Aldoart1, Serpina3f, Eln, Csf2rb, Csf2rb2, Cc17, Nov, Ccl2, Ch25h, Mt2, Socs2, Gbp4, Nptx1, Fgf23, AA467197, Gm14221, Mycl, Slc30a1, Col6a3, Mt1, Ggt5, Adam8, Plet1, and Gm7265. Genes that are downregulated after IdCil treatment are: Lsamp, Gm4322, Gng8, Igf2os, Fgfr2, Irf6, Ptprq, Fgd3, Mme, Gm36033, Itgbl1, Scd2, Scd1, Srp54a, Gm7160, Tlcd1, Scel, Smad9, Syne4, Dgki, Gm28438, Comp, Irs2, Acss2, Elmo 3, Cyp1a1, Zc2hc1c, Aass, Acaa1b, Myh11, Notum, Slc25a34, Hey2, Rubcnl, 2310014F06Rik, Brinp2, Insc, Cdkn1c, Pcsk5, Smoc1, Bcl6, Gpr160 , Pygl, Ankrd55, Cdo1, Sox9, Npr 3, Ank, Adamtsl2, Klf15, Mpl, Cfi, Trabd2b, Tnfrsfl1b, Emb, Evpl, Efcab7, Cd180, Wnt5a, Fgf2, Fam169, Ephb6, Srp54a, Gm9913, Apol9a, Kbtbd11, Gm567, Htr1b, Bdh1, Tmc8, Slc2a3, Zfp469, Camk2b, Acan, Eps8l2, Mylk3 and Sh3gl3.

FIG. 9 shows angiotensin II-treatment stimulates cardiomyocyte enlargement in vitro. Cardiomyocytes were cultured in vitro and treated with vehicle alone or AngII. (A) Bar chart represents areas of control and angiotensin II treated cardiomyocytes. Data are shown as mean±SEM. Statistical analysis by student's t-test; *P<0.05 versus control. (B) Representative images of single cardiomyocytes. Scale bar, 20 μm.

FIG. 10 shows that ldCil treatment after AAC surgery restores Myh7 transcript levels. Mice underwent either SHAM or AAC surgery and were treated with vehicle alone or ldCil 3 weeks after surgery. Bar chart represents relative Myh7 transcript expression from whole heart preparations, normalised to Gapdh. Data are shown as mean±SEM. Statistical analysis by 1-way ANOVA with Tukey's multiple comparison test post hoc. *P<0.05, n=6 hearts per group.

FIG. 11 shows KEGG and REACTOME data indicate overlap between ldCil treated AngII stimulated cardiomyocytes and non-failing human heart enriched transcriptional pathways. (A) Whole GSEA analyses underlying the data given in the heatmap of FIG. 5B. 25 common pathways where found when comparing GSEA data from mouse AngII stimulated cardiomyocytes vs control cardiomyocytes; AngII-stimulated vs AngII-stimulated plus ldCil treatment and human idiopathic cardiac myopathy vs non-failing heart. Of these 25 pathways, 12 changed concordantly: 4 concordantly upregulated and 8 downregulated pathways. (B) Whole GSEA analyses underlying the data given in the heatmap of FIG. 5C. 18 common pathways where found when comparing GSEA data from mouse AngII stimulated cardiomyocytes vs control cardiomyocytes; AngII-stimulated vs AngII-stimulated plus ldCil treatment and human ischaemic cardiac myopathy vs non-failing heart. Of these 18 pathways, 6 changed concordantly. Together, these data indicate that ldCil treatment reverses molecular signatures that are associated with human failing heart back to that of human non-failing heart.

FIG. 12 shows protein validation of common differentially expressed TCA-cycle enzymes in non-failing human and ldCil treated AngII-stressed cardiomyocytes. Western blot and densitometric analysis. (A) Protein validation of RNA-Seq data hits showing upregulation of TCA cycle enzymes: aconitase, pyruvate dehydrogenase and succinate dehydrogenase, in dilated and ischaemic human cardiomyopathy whole tissue protein lysates vs. non-failing human heart protein lysates. n, 4-6 human tissue sample lysates/tissue type. Data are shown as mean±SEM. Students t-test, *P<0.05, **P<0.01. (B) Low dose Cilengitide treatment of AngII-stressed mouse cardiomyocytes rescues the elevated expression of the TCA cycle enzyme pyruvate dehydrogenase found to be upregulated in human failing hearts. Data are shown as mean±SD. N, 2-3 mouse cardiomyocyte preparations. Students t-test, P<0.05.

### BRIEF DESCRIPTION OF THE SEQUENCES

SEQ ID NOs: 1-46 as shown in the description and sequence listing are amino acid sequences of peptides and prodrugs.

### DETAILED DESCRIPTION

It is to be understood that different applications of the disclosed products and methods may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural referents unless the content clearly dictates otherwise. Thus, for example, reference to “an αvβ3- and/or αvβ5-integrin targeting agent” includes two or more such agents and the like.

All publications, patents and patent applications cited herein, whether supra or infra, are hereby incorporated by reference in their entirety.

### Methods of Prevention or Treatment

The invention provides methods of preventing or treating a vascular condition or heart failure in a patient, comprising administering an αvβ3- and/or αvβ5-integrin targeting agent to the patient.

### Agent

The αvβ3- and/or αvβ5-integrin targeting agent may be any agent that targets αvβ3-and/or αvβ5-integrins in a manner suitable to prevent or treat a vascular condition or heart failure, by any means. The agent may promote angiogenesis or endothelial cell activation. The agent may target αvβ3- and/or αvβ5-integrins to promote cardiac or vascular function. The agent may inhibit or reduce cardiac hypertrophy (such as cardiomyocyte hypertrophy) or decrease heart:body weight ratio. Administration of the agent may alter or prevent changes to one or more parameters of cardiac function, including fractional shortening, ejection fraction and left ventricular internal dimension in systole. The above changes typically occur independently of changes in mean arterial blood pressure. The agent may increase fractional shortening and/or ejection fraction or prevent a decrease in fractional shortening and/or ejection fraction. The agent may decrease or prevent an increase in left ventricular internal dimension in systole. Administration of the agent may enhance or prevent a decrease in cardiac blood vessel density. The agent may increase vascular perfusion, such as cardiac vascular perfusion. The agent may increase microvascular blood flow. The agent may increase vasculature, for example in any ischemic disease.

The above functional properties may be readily evaluated by the skilled person, including by use of the mouse model and cell culture assay described in the Examples, and using techniques known in the art, such as echocardiography.

The agent may target αvβ3- and/or αvβ5-integrins so as to induce altered signalling and/or gene expression. The agent may give rise to upregulation of one or more proangiogenic or cardioprotective regulators, such as one or more of CSF2RB, Eln, Nov, CCL7, Mt2, Socs2, Fgf23, Slc30a1, Col6a3, Mt1, Ggt5 and Adam8. The agent may enhance activation and recycling of the pro-angiogenic receptor VEGF-receptor 2 (27). The agent may upregulate one or more genes representative of enhanced angiogenesis and/or myocardial response to injury/pressure overload, such as one or more genes involved in mitosis, DNA repair or cell cycle regulation. The agent may thus restore a signalling pattern indicative of normal heart or vascular function, such as normal cardiomyocyte and/or cardiac endothelial cell function or normal vascular endothelial cell function. Normal heart function is typically assessed with reference to a non-failing heart or a non-hypertrophic heart. The agent may reduce or prevent increase in activity of the TCA cycle or of one or more oxidative stress pathways. The agent may increase or prevent decrease in activity of insulin, PI3K, Akt and/or NGF signalling pathways.

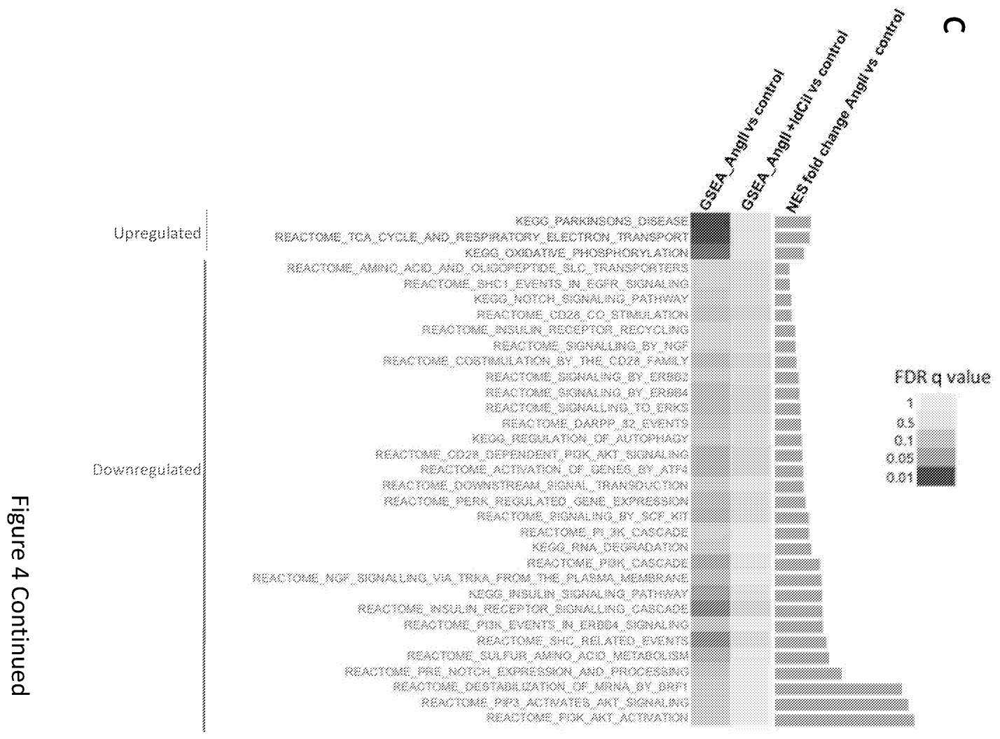

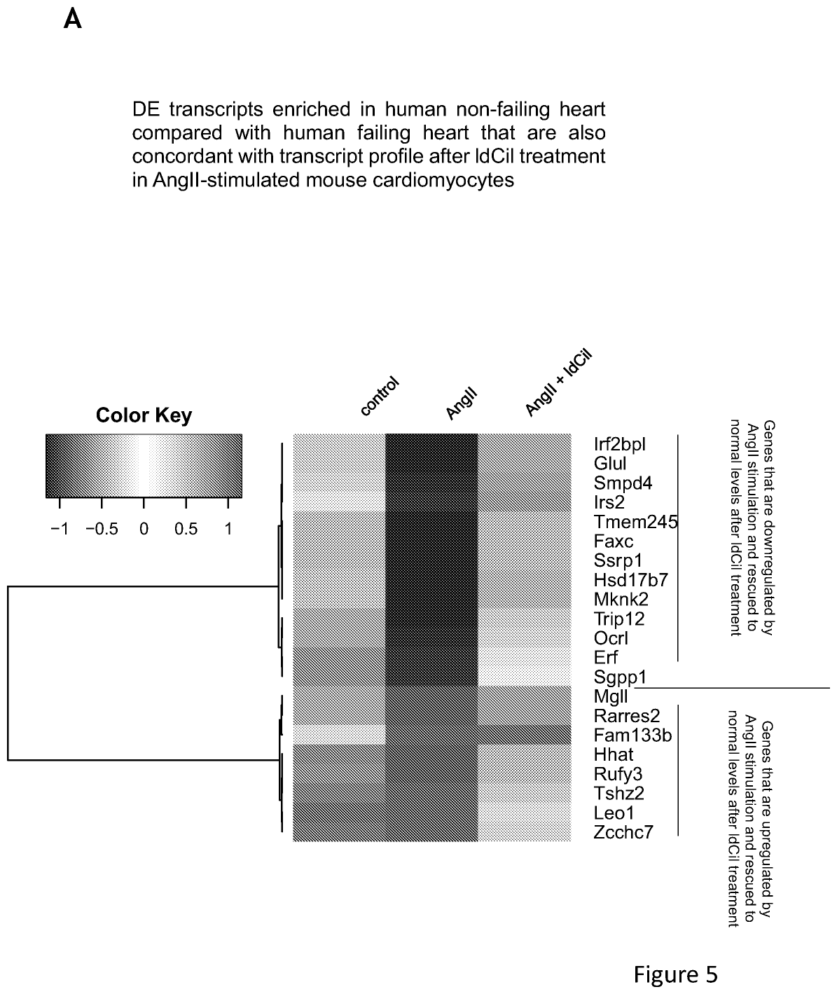

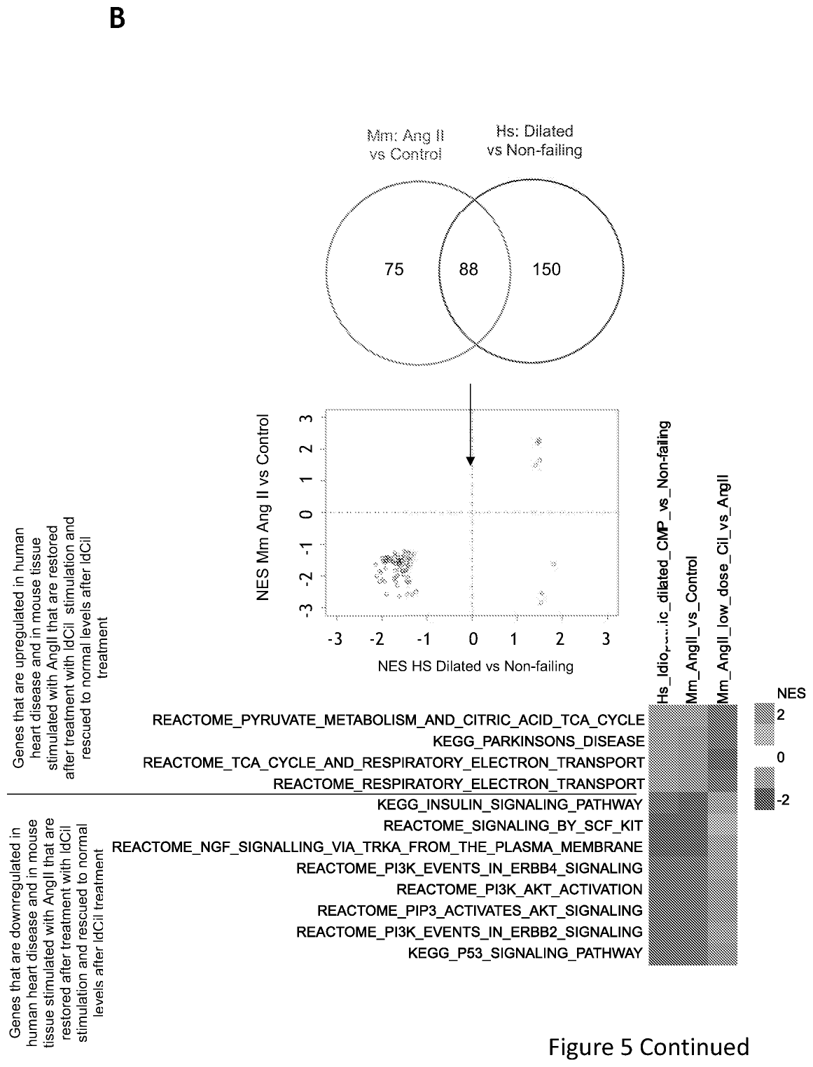

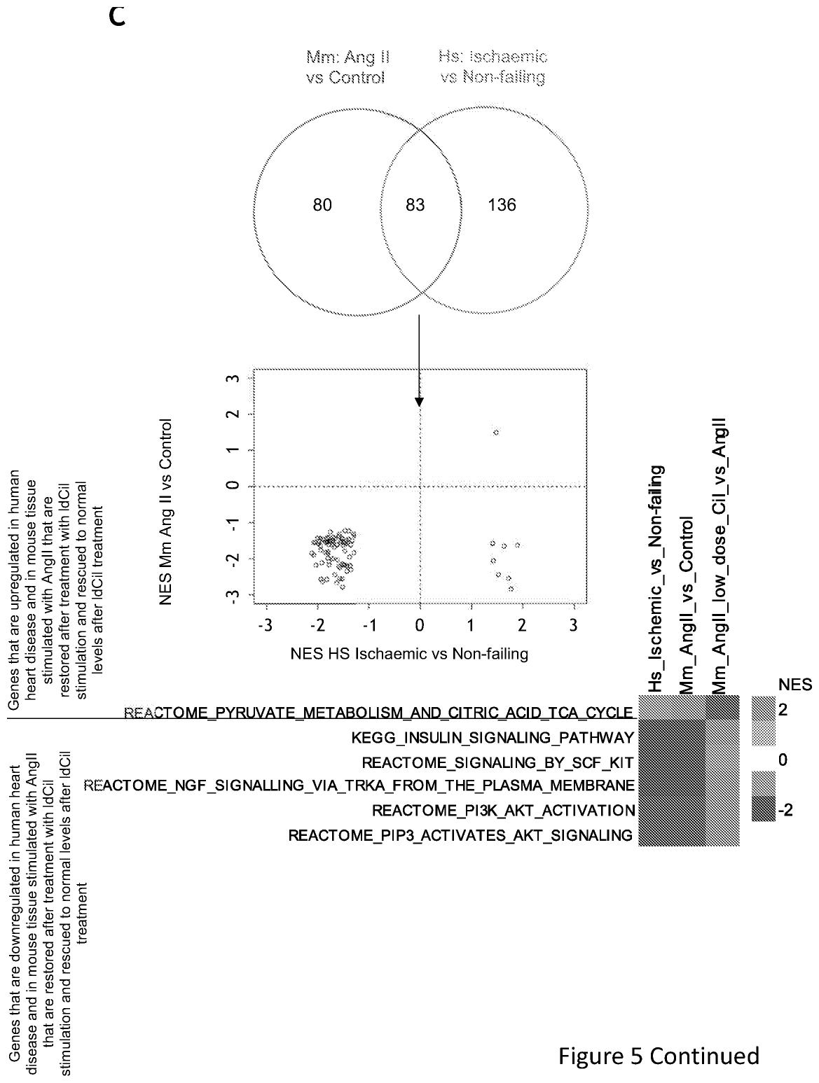

The agent may reduce or prevent increase in expression of one or more genes whose upregulation is associated with hypertrophy or of one or more genes upregulated in response to AngII treatment of cardiomyocytes, such as one or genes involved in cardiac remodelling or other hypertrophic markers, for example Myh7. The agent may increase or prevent reduction in expression of one or more, such as at least two, at least three, at least four, or at least five of Irf2bp1, Glu1, Smpd4, Irs2, Tmem245, Faxc, Ssrp1, Hsd17b7, Mknk2, Trip12, Ocr1, Erf, and Sgpp1, which are genes concordantly downregulated in mouse cardiomyocytes on AngII treatment (and reduced by an αvβ3- and/or αvβ5-integrin targeting agent) and in human heart failure, as shown in FIG. 5A. The agent may reduce or prevent increase in expression of one or more, such as at least two, at least three, or at least four of Mg11, Rarres2, Fam133b, Hhat, Rufy3, Tshz2, Leo1 and Zcchc7, which are genes concordantly upregulated in mouse cardiomyocytes on AngII treatment (and reduced by an αvβ3- and/or αvβ5-integrin targeting agent) and in human heart failure, as shown in FIG. 5A. In all of the above aspects, the agent may restore expression of the relevant gene to a level similar to normal vasculature or heart.

The above effects on gene expression and signalling may be readily evaluated by the skilled person, including by use of the mouse model and in vitro cardiomyocyte assay (optionally with AngII treatment) as described in the Examples.

Typically, the targeting agent induces altered signalling and/or gene expression but does not affect integrin adhesive function. The agent may thus be an integrin activator.

The targeting agent may target αvβ3- and/or αvβ5-integrin and optionally further integrins. The agent thus preferably binds integrins which are expressed in vascular or cardiac endothelial cells or cardiomyocytes. Typically, the agent targets αvβ3-integrin and optionally may also target αvβ5-integrin. Preferably, the agent is selective for αvβ3-integrin (and optionally also for αvβ5-integrin) over other integrins and does not display high binding affinity for platelet integrins such as αIIbβ3. In some embodiments, the targeting agent may also target one or more of integrins αvβ6, αvβ8 and αvβ1, typically at reduced affinity as compared to αvβ3.

The agent may be any type of molecule, such as a small molecule ligand (such as an organic compound of less than 5 kDa), a peptide, a protein, an antibody, a polynucleotide, or an oligonucleotide.

Typically, the agent is a small molecule ligand, a peptide, a protein, or an antibody. Such an agent typically specifically binds directly to αvβ3- and/or αvβ5-integrin, but agents that target αvβ3- and/or αvβ5-integrin indirectly to provide for the effects described above may also be used. An agent specifically binds to a target (αvβ3- and/or αvβ5-integrin) when it binds with preferential or high affinity to that target but does not substantially bind, does not bind or binds with only low affinity to other targets. For instance, an antibody “specifically binds” a target protein when it binds with preferential or high affinity to that target protein but does not substantially bind, does not bind or binds with only low affinity to other human protein.

An antibody binds with preferential or high affinity if it binds with a Kd of 1×10−7 M or less, more preferably 5×10−8 M or less, more preferably 1×10−8 M or less or more preferably 5×10−9 M or less. An antibody binds with low affinity if it binds with a Kd of 1×10−6 M or more, more preferably 1×10−5 M or more, more preferably 1×10−4 M or more, more preferably 1×10−3 M or more, even more preferably 1×10−2 M or more.

The antibody may be, for example, a monoclonal antibody, a polyclonal antibody, a single chain antibody, a chimeric antibody, a bispecific antibody, a CDR-grafted antibody or a humanized antibody. The antibody may be an intact immunoglobulin molecule or a fragment thereof such as a Fab, F(ab′)₂ or Fv fragment.

Preferably, the agent is a peptide. The term “peptide” as used herein is meant to encompass a chain of natural (genetically encoded), non-natural and/or chemically modified amino acid residues. The amino acid residues are represented throughout the specification and claims by either one or three-letter codes, as is commonly known in the art.

The amino acids in the sequences of peptides described herein are typically represented by a single letter as known in the art, wherein a small letter represents the corresponding amino acid in the D configuration (and a capital letter the L configuration). An asterisk symbol followed by a letter means that the corresponding amino acid is N-methylated.

Peptide described herein may be 4, 5, 6, 7, 8, 9, 10, 11, 12, 13, 14, 15, 16, 17, 18, 19, or 20 amino acids in length. Peptides described herein may be 4 to 10, 5 to 19, 5 to 18, 15 to 17, 5 to 16, 5 to 15, 5 to 14, 5 to 13, 5 to 12, 5 to 11, 5 to 10, 5 to 9, 5 to 8, or 5 to 7 amino acids in length.

Peptides described herein are typically cyclized. The terms “cyclo” or “cyclic” are used herein interchangeably and intend to indicate that the peptide is cyclized. Any type of cyclization may be applied, including but not limited to: head-to-tail cyclization, side chain to side chain, sidechain to terminal, disulfide bridge, or backbone cyclization, preferably head-to-tail cyclization. In some embodiments, the peptides comprise head-to-tail cyclization, namely having a covalent bond between the atoms of the amino terminal amino group and the carboxyl terminus of the peptide. A “c” letter followed by brackets delineating a peptide sequence means that said peptide is cyclic. Principles of design of cyclic peptides, including integrin-targeting cyclic peptides are well known in the art, and described for example in Weide et al (Topics in Curr Chemistry 2007, 272, 1-40) and Kessler (ACIE 1982, 21, 512-523).

Typical cyclic peptides targeting αvβ3- and/or αvβ5-integrin comprise the sequence motif RGD. R, and/or D may be L- or D-amino acids. Amino acids in the motif may be modified provided that this is compatible with activity of the peptide as a targeting agent e.g. the introduction of the modification should not impair the ability of the cylic peptide to target αvβ3- and/or αvβ5-integrin. For example, the length of the aliphatic chain between the alpha carbon of the amino acid to the guanidinium functional group in Arg may be altered.

The ability of a peptide to target αvβ3- and/or αvβ5-integrin can be determined using various methods. For example, the ability of peptide described herein to target αvβ3- and/or αvβ5-integrin may be determined by ELISA (see e.g. Kapp et al. Sci Rep. 2017; 7:39805), cellular assays or force field microscopy.

A cyclic peptide comprising the sequence motif RGD is typically about five to about eight amino acids in length. Such a cyclic peptide may be five, six, seven or eight amino acids in length, such as five to seven, five to six, six to seven or six to eight amino acids in length. Preferably, the peptide is a pentapeptide or a hexapeptide. The peptide may comprise two β-turns.

The above cyclic peptides may comprise at least one N-alkylated (such as N-methylated) amino acid residue, such as at least two, or at least three N-alkylations or N-methylations. The N-alkylation (such as N-methylation) may assist in constraining peptide conformation and/or increase metabolic stability. Said peptides may comprise 4 or 5 N-alkylations or N-methylations. The cyclic peptide may be a hexapeptide comprising the above specified numbers of N-alkylations or N-methylations. The N-alkylations or N-methylations may be in a position selected from the group consisting of (1,5), (1,6), (3,5), and (5,6).

The above cyclic peptides may comprise at least one D-amino acid. The cyclic peptide may be an N-methylated cyclic pentapeptide or hexapeptide comprising at least one amino acid in the D configuration. Such a peptide may comprise at least one alanine residue in the D configuration, at least one valine residue or at least one phenylalanine residue in the D configuration. The amino acid in D configuration may be at position number 1 in the peptide or in the i+1 position of a βII′ turn.

The above cyclic peptides may comprise at least one β-amino acid or γ-amino acid. The above cyclic peptides may additionally comprise at least one hydrophobic amino acid residue (such as Pro, Ile, Leu, Val, Phe, Trp, Tyr, Met), preferably selected from Val or Phe.

The peptide may be cilengitide (c(f*VRGD) or a derivative or analogue thereof, including derivatives or analogues where amino acids are changed to the L- or D-configuration, where f and/or V are substituted for alternative hydrophobic amino acids (such as Pro, Ile, Leu, Val, Phe, Trp, Tyr, Met) or for Gly, or where the peptide comprises an alternative N-alkylation to the specified N-Me or additional N-alkylations or N-methylations. Typically, a D-amino acid or Gly is provided at the i+1 position of a βII′ turn (Muller et al. Proteins: Structure, Function, and Genetics 1993, 15, 235-251).

The peptide may be one of the following peptides or a derivative or analogue thereof:

(SEQ ID NO: 1)
a. *rGDA*AA;
(SEQ ID NO: 13)
b. *rGDAA*A;
(SEQ ID NO: 2)
c. *aRGDA*A;
(SEQ ID NO: 3)
d. rG*DA*AA;
(SEQ ID NO: 4)
e. rGDA*A*A;
(SEQ ID NO: 5)
f. *vRGDA*A;
(SEQ ID NO: 6)
g. *fRGDA*A;
(SEQ ID NO: 7)
h. *rGDA*AV;
(SEQ ID NO: 8)
i. *rGDA*AF;
(SEQ ID NO: 17)
j. LPPFRGDLA;
(SEQ ID NO: 18)
k. LPPGLRGD;
(SEQ ID NO: 19)
l. aAAAAA;
or
(SEQ ID NO: 20)
m. f*VRGD.

wherein * represents N-methylation of the following amino acid, R is arginine, G is glycine, D is aspartic acid, r is arginine in the D configuration, A is alanine, a is alanine in the D configuration, V is valine, v is valine in the D configuration, F is phenylalanine, and f is phenylalanine in the D configuration.

As discussed above the peptides of the invention are typically cyclized. Thus, the invention also relates to the cyclic peptides of SEQ ID NOS: 9 to 12, 24 to 31 and 36 to 46. By way of example, non-cyclized versions of the peptides of SEQ ID NOS: 9 to 12 are also described as SEQ ID NOS: 32-35.

The peptides of a. and c. to g. are also described in WO2019058374 and shown to exhibit improved specific properties such as high affinity, high selectivity and metabolic stability, high stability and suitability for oral administration, particularly when formulated as a prodrug. Preferred peptides were optimized to include a D-amino acid at a defined position and to include Phe or Val. A particularly preferred peptide is peptide f. (SEQ ID NO: 5).

Derivatives or analogues of the above peptides of a. to h. include peptides where an alternative D-amino acid or Gly is included at position 1 and/or wherein one or more alanine residues are substituted for other amino acids, typically for other uncharged amino acids, amino acids that do not have bulky side chains (for example Ile, Leu, Val, Met) and/or non-natural amino acids. Derivatives or analogues may include an alternative N-alkylation to the specified N-Me or additional N-alkylations or N-methylations. Where a peptide of a. to .f includes Phe or D-Phe, this may be substituted for Tyr or D-Tyr. The derivatives or analogues typically include a D-amino acid or Gly at the i+1 position of a βII′ turn (as described in Muller et al. Proteins: Structure, Function, and Genetics 1993, 15, 235-251), or at position 1 in the peptide.

Derivatives or analogues of any of the above peptides typically preserve or enhance activity of the peptide as a targeting agent, and may be validated for activity by the skilled person using methods known in the art and described in the Examples.

Also described herein are peptidomimetics of any of the above peptides. Principles for design of peptidomimetics are well known in the art. Including in the context of integrin-binding agents as described for example in Kapp et al. Sci Rep.: 2017 7:39805, Neubauer et al. J Med Chem 2014, 57, 3410 and Intervoll et al. Bioorg Med Chem Let 2006, 16, 6190-93.

Also described herein are salts of the peptides and peptidomimetics described described above, and analogs and chemical derivatives of the peptides and peptidomimetics.

As used herein the term “salts” refers to both salts of carboxyl groups and to acid addition salts of amino or guanido groups of the peptide molecule. Salts of carboxyl groups may be formed by means known in the art and include inorganic salts, for example sodium, calcium, ammonium, ferric or zinc salts, and the like, and salts with organic bases such as salts formed for example with amines such as triethanolamine, piperidine, procaine, and the like. Acid addition salts include, for example, salts with mineral acids such as, for example, acetic acid or oxalic acid. Salts describe here also ionic components added to the peptide solution to enhance hydrogel formation and/or mineralization of calcium minerals. Pharmaceutically acceptable salts include those formed with free amino groups such as those derived from hydrochloric, phosphoric, acetic, oxalic, tartaric acids, and the like, and those formed with free carboxyl groups such as those derived from sodium, potassium, ammonium, calcium, ferric hydroxides, isopropylamine, triethylamine, 2-ethylamino ethanol, histidine, procaine, and the like.

The peptides may be produced by any method known in the art, including recombinant and synthetic methods. Synthetic methods include solid phase synthesis, partial solid phase synthesis, fragment condensation, or classical solution synthesis. Solid phase peptide synthesis procedures are well known to one skilled in the art and described, for example by John Morrow Stewart and Janis Dillaha Young, Solid Phase Polypeptide Syntheses (2nd Ed., Pierce Chemical Company, 1984), Biron & Kessler (J. Org. Chem. 2005, 70, 5183-5189) and Chatterjee et al (Nature protocols, 2012, 7 (3), 432-444). In some embodiments, synthetic peptides are purified by preparative high-performance liquid chromatography (Creighton T. (1983) Proteins, structures and molecular principles. WH Freeman and Co. N.Y.).

The peptides may also be provided in the form of a prodrug, or is provided as a conjugate or fusion protein. The term “prodrug” as used herein refers to an inactive or relatively less active form of an active agent that becomes active through one or more metabolic processes in a subject. Prodrugs of peptides disclosed herein may comprise the modification of amino acids and/or amino acid residues to include an ester(s) and/or carbamate(s) of primary alcohols. In some embodiments and generally, amino side chains having amine moieties are modified into carbamates having —NHCO₂R moieties; whereas amino side chains having carboxylate moieties are modified into esters having —CO₂R moieties.

Prodrugs of the above peptides may have a net neutral charge, preferably being uncharged at physiological pH. The prodrug may comprise at least one moiety that reduces net charge of the peptide. For example, a peptide (such as an N-methylated cyclic hexapeptide) may be linked to at least one molecule that masks the charge of the amino acids in the peptide. The peptide may comprise a permeability enhancing moiety coupled to the peptide's sequence directly or through a spacer or linker. The spacer or linker may comprise a protease-specific cleavage site.

The prodrug may comprise at least one of the following moieties or groups of moieties (also described as permeability enhancing moieties):

a —CO2R moiety, wherein R is alkyl;

(ii) a penta, hexyl or heptaoxycarbonyl moiety, optionally linked to an arginine residue;

(iii) a methyl ester moiety (OMe) or other alkyl ester moiety, such as a methyl or alkyl ester of Asp;

(iv) penta, hexyl or heptaoxycarbonyl and ester (OMe) moieties; and/or

(v) two penta, hexyl or heptaoxycarbonyl moieties, such as two hexyloxycarbonyl moieties.

The at least one —CO₂R moiety may be covalently linked to a nitrogen atom of at least one amino acid side chain (such as of an arginine side chain) of the peptide, preferably an N-methylated cyclic hexapeptide, such as the RGD, cyclohexapeptides described by A. Schumacher-Klinger et al. (Molecular Pharmaceutics 2018, 15. 3468-3477).

The prodrug may comprise the moiety:

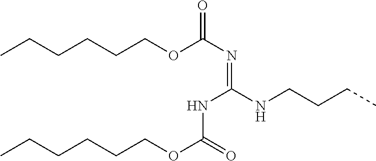

where the broken line indicates a covalent bond between the moiety and the backbone of the peptide, such as an N-methylated cyclic hexapeptide. According to some embodiments, the broken line represents a covalent bond between the moiety and an a-carbon of the peptide, such as an N-methylated cyclic hexapeptide.

R of the above moiety may be a primary alkyl group. R may be n-hexyl or any alkyl group, such as n-C₁₄H₂₉ (myristyl).

The permeability-enhancing moiety may be an oxycarbonyl moiety such as a penta, hexyl or heptaoxycarbonyl (Hoc) moiety. Hoc in all structures designates a hexyloxycarbonyl residue having the structure:

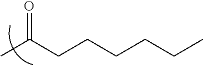

The guanidine group of Arg of the peptide may be masked with a Hoc or two Hoc moieties.

In another prodrug modification, the peptide may comprise a methyl ester moiety (OMe) or other alkyl ester moiety, such as a methyl or alkyl ester of Asp. The peptide may comprise at least one side chain having the formula CH₂COOMe.

In specific aspect, the prodrug of the peptide may have the formula:

(SEQ ID NO: 9)
c(*vR(Hoc)₂GD(OMe)A*A),
(SEQ ID NO: 10)
c(*aR(Hoc)₂GD(OMe)A*A),
(SEQ ID NO: 11)
c(*r(Hoc)₂GD(OMe)A*AA)
(SEQ ID NO: 12)
c(r(Hoc)₂GD(OMe)A*A*A),
(SEQ ID NO: 14)
*fR(Hoc)₂GD(OMe)A*A,
(SEQ ID NO: 15)
LPPFR(Hoc)₂GDLA,
(SEQ ID NO: 16)
LPPGLR(Hoc)₂GD,
(SEQ ID NO: 21)
*vR(Hoc)GD(OMe)A*A,
(SEQ ID NO: 22)
a*AA(Hoc)₂AAA
or
(SEQ ID NO: 23)
f*VR(Hoc)₂GD.

Further information on provision of preferred αvβ3- and/or αvβ5-integrin targeting peptides is provided in WO2019058374 and Weinmüller et al (Angew. Chem. Int. Ed. 2017, 56, 16405-16409), the disclosures of which (including of all designed peptides described in the documents) are incorporated by reference herein.

### Method of Preventing or Treating Vascular Condition or Heart Failure

The αvβ3- and/or αvβ5-integrin targeting agent is used in a method for preventing or treating a vascular condition or heart failure in a patient. The agent has functional properties as described above that are particularly suitable for these diseases, which involve defects in angiogenesis or vascular or cardiac function. The agent may thus be used to treat any vascular condition and any form of heart failure.

The patient is typically human. However, the patient may be another mammalian animal, such as a commercially farmed animal, such as a horse, a cow, a sheep, a fish, a chicken or a pig, a laboratory animal, such as a mouse or a rat, or a pet, such as a guinea pig, a hamster, a rabbit, a cat or a dog.

Heart failure is a chronic disease which has varying levels of severity and may worsen over time. Heart failure typically comprises reduced cardiac function, such as reduced ability to pump blood and maintain normal blood flow. Heart failure prevented or treated in accordance with the invention may comprise congestive heart failure, hypertrophic, dilated or ischaemic cardiomyopathy, and/or coronary microvascular disease. The heart failure may comprise cardiomyocyte hypertrophy. The heart failure may be associated with an increased heart:body weight ratio, reduced fractional shortening, reduced ejection fraction, and/or increased left ventricular internal dimension in systole. The heart failure may comprise reduced cardiac blood vessel density, reduced cardiac vascular perfusion, and/or reduced coronary microvascular blood flow.

A vascular condition or disease is any condition or disease involving dysfunction of blood vessels, typically characterised by reduced blood flow or perfusion, such as reduced microvascular blood flow, or restricted blood flow to tissues. The vascular condition or disease may comprise endothelial cell dysfunction.

The vascular condition or disease may be cardiovascular disease, peripheral vascular disease, or ischemia. Cardiovascular disease (CVD) refers to a class of diseases that involve the heart or blood vessels. Cardiovascular diseases involving the blood vessels are also known as vascular diseases. The disease or condition may be selected from any of coronary artery disease, coronary heart disease, ischemic heart disease, peripheral arterial disease, cerebrovascular disease, stroke, mini-stroke, renal artery stenosis, aortic aneurysm, arteriosclerosis, atherosclerosis, myocardial ischemia, angina, ischemic vascular disease, myocardial infarction, aneurysm, restenosis, ischemia/reperfusion injury, sepsis, a chronic wound, and pulmonary hypertension. The disease or condition may be any cardiovascular disease that involves the heart. The disease or condition may be selected from any of hypertensive heart disease, secondary to high blood pressure, hypertension, heart failure, pulmonary heart disease, cardiac dysrhythmias, abnormalities of heart rhythm, inflammatory heart disease, endocarditis, inflammatory cardiomegaly, myocarditis, valvular heart disease, and rheumatic heart disease. The disease or condition may be hypercholesterolemia, such as familial hypercholesterolemia.

The vascular condition or heart failure may exhibit overexpression of αvβ3- and/or αvβ5-integrins, preferably overexpression of αvβ3-integrins. The heart failure may preferably be ischemic or dilated heart failure exhibiting overexpression of αvβ3- and/or αvβ5-integrins, preferably overexpression of αvβ3-integrins.

The patient may be previously diagnosed with heart failure or a vascular condition (for example by physical examination, symptom history, or echocardiography) or established as being at risk of heart failure or a vascular condition by one or more risk indicia such as gender, age, family history, weight, body mass index, diet or lifestyle. The individual may be over the age of 40; according to NHS Guidelines, people over 40 should have their estimate of CVD risk reviewed regularly. The individual may have a family history of early cardiovascular disease—for example, having a father or brother that developed heart disease or had a heart attack or stroke before the age of 55, or a mother or sister that had such a condition before the age of 65. The individual may be overweight or obese. The individual may have high blood pressure, high cholesterol or diabetes. The patient may have experienced a myocardial infarction or cardiac arrest.

The patient may exhibit one or more symptoms of a vascular condition or heart failure, such as shortness of breath, swollen legs, feet, ankles, stomach and/or lower back, or tiredness or weakness. Administration of the agent may relieve one or more of these symptoms by treating the underlying cause.

The invention also provides medical uses corresponding to the methods of prevention or treatment described above and incorporating any feature described above for the methods of the invention. The invention thus provides an αvβ3- and/or αvβ5-integrin targeting agent for use in a method of preventing or treating a vascular condition or heart failure in a patient. The invention also provides use of an αvβ3- and/or αvβ5-integrin targeting agent in the manufacture of a medicament for preventing or treating a vascular condition or heart failure.

### Pharmaceutical Composition

The agents described herein may be formulated in pharmaceutical compositions. The above methods and medical uses typically comprise administration of a pharmaceutical composition. The compositions may comprise, in addition to the therapeutically active ingredient(s), a pharmaceutically acceptable excipient, carrier, diluent, buffer, stabiliser or other materials well known to those skilled in the art. Such materials should be non-toxic and should not interfere with the efficacy of the active ingredient. The pharmaceutical carrier or diluent may be, for example, an isotonic solution.

The term “pharmaceutically acceptable” means approved by a regulatory agency such as a Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic compound is administered. Such pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents such as acetates, citrates or phosphates. Antioxidants such as ascorbic acid or sodium bisulfate; chelating agents such as ethylenediaminetetraacetic acid; and agents for the adjustment of tonicity such as sodium chloride or dextrose are also envisioned.

The precise nature of the carrier or other material may depend on the route of administration, e.g. oral, intravenous, cutaneous or subcutaneous, nasal, intramuscular and intraperitoneal routes. Examples of suitable pharmaceutical carriers are described in “Remington's Pharmaceutical Sciences” by E. W. Martin. Such compositions will contain a therapeutically effective amount of the peptides according to the invention, preferably in a substantially purified form, together with a suitable amount of carrier so as to provide the form for proper administration to the subject. Examples of suitable compositions and methods of administration are provided in Esseku and Adeyeye (2011) and Van den Mooter G. (2006).

For example, solid oral forms may contain, together with the active substance, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, gum arabic, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tabletting, sugar-coating, or film-coating processes.

Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10% to 95% of active ingredient, preferably 25% to 70%. Where the pharmaceutical composition is lyophilised, the lyophilised material may be reconstituted prior to administration, e.g. a suspension. Reconstitution is preferably effected in buffer.

Capsules, tablets and pills for oral administration to an individual may be provided with an enteric coating comprising, for example, Eudragit “S”, Eudragit “L”, cellulose acetate, cellulose acetate phthalate or hydroxypropylmethyl cellulose.

Liquid dispersions for oral administration may be syrups, emulsions or suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with the active substance, a pharmaceutically acceptable carrier, e.g. sterile water, olive oil, ethyl oleate, glycols, e.g. propylene glycol, and if desired, a suitable amount of lidocaine hydrochloride.

Solutions for intravenous administration or infusion may contain as carrier, for example, sterile water or preferably they may be in the form of sterile, aqueous, isotonic saline solutions.

For suppositories, traditional binders and carriers may include, for example, polyalkylene glycols or triglycerides; such suppositories may be formed from mixtures containing the active ingredient in the range of 0.5% to 10%, preferably 1% to 2%.

The pharmaceutical composition (such as a composition for oral administration) may comprise absorption enhancers. The pharmaceutical composition may comprise lipids. The pharmaceutical composition may comprise self nano-emulsifying drug delivery systems (SNEDDS) or Pro-NanoLiposphere (PNL). The term SNEDDS (self nano-emulsifying drug delivery systems) as used herein refers to anhydrous homogeneous liquid mixtures, composed of oil, surfactant, drug, and/or cosolvents, which spontaneously form transparent nanoemulsion. The term PNL (Pro-NanoLiposphere) as used herein refers to a delivery system based on a solution containing the drug, triglyceride, phospholipid, surfactants, and a water miscible organic solvent.

The pharmaceutical composition may comprise curcumin, resveratrol and/or piperine. The pharmaceutical composition may comprise Resveratrol-PNL and/or Piperine-PNL. The pharmaceutical composition may additionally or alternatively comprise elements that reduce intra-enterocyte metabolism by CYP3A4 enzymes and/or reduce P-gp efflux activity.

### Administration and Dose

The agent or pharmaceutical composition comprising said agent is administered to treat or prevent a vascular condition or heart failure. Administration is typically in a “prophylactically effective amount” or a “therapeutically effective amount” (as the case may be, although prophylaxis may be considered therapy), this being sufficient to show benefit to the individual, e.g. an effective amount to prevent or delay onset of the disease or condition, to ameliorate one or more symptoms, to induce or prolong remission, or to delay relapse or recurrence.

The agent or pharmaceutical composition may be administered by any route, including an oral, intravenous, cutaneous or subcutaneous, nasal, intramuscular or intraperitoneal route.

The dose may be determined according to various parameters, especially according to the substance used; the age, sex, weight and condition of the individual to be treated; the route of administration; and the required regimen. A physician will be able to determine the required route of administration and dosage for any particular individual. A typical daily dose is from about 0.1 to 50 mg per kg of body weight dependent on the conditions mentioned above. The dose may be provided as a single dose or may be provided as multiple doses, for example taken at regular intervals. The dosage can be administered, for example, in weekly, biweekly, monthly or bimonthly regimens. Examples of the techniques and protocols mentioned above can be found in Remington's Pharmaceutical Sciences, 20th Edition, 2000, pub. Lippincott, Williams & Wilkins.

A general dose amount described in the art for administration of pharmaceutical compositions comprising peptides ranges from about 0.1 μg/kg to about 20 mg/kg body weight. The amount of the active ingredient may be in the range of from about 10 to 5000 μg/kg.

The peptides described herein, in particular any of the cyclic peptides of about five to about eight amino acids in length comprising the sequence RGD as described above, such as cilengetide or the peptides and prodrugs of SEQ ID NOs: 1-23 and derivatives and analogs thereof, and peptidomimetics thereof, are administered at a dose effective to induce one or more of the functional properties of the agents as described above, and to prevent or treat the vascular condition or heart failure. The dose is typically effective to alter αvβ3- and/or αvβ5 integrin signaling without inhibiting integrin adhesion. The amount of the peptides which will be effective in the treatment of a particular disorder or condition will depend on the nature of the disorder or condition, and can be determined by standard clinical techniques. In addition, in vitro assays may optionally be employed to help identify optimal dosage ranges. The precise dose to be employed in the formulation will also depend on the seriousness of the disease or disorder, and should be decided according to the judgment of the practitioner and each patient's circumstances.

Toxicity and therapeutic efficacy of the peptides described herein can be determined by standard pharmaceutical procedures in cell cultures or experimental animals, e.g., by determining the IC50 (the concentration which provides 50% inhibition) and the LD50 (lethal dose causing death in 50% of the tested animals) for a subject compound. The data obtained from these cell culture assays and animal studies can be used in formulating a range of dosage for use in humans. The dose amount for humans may be determined by comparison with a mouse dose amount of about 50 μg/kg to about 400 μg/kg, such as about 50 μg/kg to about 300 μg/kg, about 50 μg/kg to about 200 μg/kg, or about 50 μg/kg to about 200 μg/kg.

The dose amount in humans may also be selected based on any of the dose ranges described below. A peptide may be administered at a dose ranging from about 0.1 mg/kg to about 10 mg/kg of the subject weight. A peptide may be administered at a dose ranging from about 0.01, 0.05, 0.1, 0.5, 0.7, 1, or 2 mg/kg of the subject weight, to about 0.05, 0.1, 0.5, 0.7, 1, 2, 5, 10, 15, 20, 50, 100, 250, or 500 mg/kg of the subject weight. A peptide may be administered at a dose ranging from 0.1, 1, 10, 20, 30, 50, 100, 200, 400, 500, 700, 900 or 1000 ng/kg of the subject weight, to about 100, 200, 400, 500, 700, 900, 1000, 1200, 1400, 1700, or 2000 ng/kg of the subject weight. A peptide may be administered at a dose ranging from about 20 ng/kg to about 100 ng/kg of the subject weight.

The peptide may be orally administered at a dose ranging from about 0.001 mg/kg to about 500 mg/kg of the subject weight, for example from about 0.1 mg/kg to about 500 mg/kg of the subject's body weight.

### Combination

The αvβ3- and/or αvβ5-integrin targeting agent may be administered in the methods and medical uses of the invention combination with at least one additional agent suitable for preventing or treating a vascular condition, heart failure or symptoms of heart failure. Any such agent known in the art may be administered in the above combination. Such additional agents may be selected for example from antihyptertensive drugs, ACE (angiotensin converting enzyme) inhibitors, ARB (angiotensin receptor blockers), beta blockers, neprilysin inhibitors, diuretics, hydralazine, and nitrovasodilators.

In a related aspect, the invention also provides a combination of an αvβ3- and/or αvβ5-integrin targeting agent and at least one additional agent suitable for preventing or treating a vascular condition, heart failure or symptoms of heart failure, as a product per se. The additional agent may be any of the specific agents described above. In some embodiments, the agent is not verapamil. The above combination may take the form of a composition comprising the αvβ3- and/or αvβ5-integrin targeting agent and at least one additional agent, or a product comprising different dose forms of the targeting agent and additional agents. The combination may be a kit comprising the αvβ3- and/or αvβ5-integrin targeting agent and at least one additional agent, optionally with instructions for administration in a method of the invention. The additional agent may be for administration simultaneously or sequentially with the αvβ3- and/or αvβ5-integrin targeting agent.

### EXAMPLES

The following Examples are provided to illustrate the invention.

### Materials and Methods

### Human Heart Tissue

Procurement of human myocardial tissue was performed under protocol ethical regulations and approved by Institutional Review Boards at the University of Pennsylvania and the Gift-of-Life Donor Program (Pennsylvania, USA). Failing human hearts were procured at the time of orthotopic heart transplantation at the Hospital of the University of Pennsylvania following informed consent from all participants. Non-failing hearts were obtained at the time of organ donation from cadaveric donors, with consent obtained from next-of-kin. In all cases, hearts were arrested in situ using ice-cold cardioplegia solution and transported on wet ice. Whole hearts and dissected left ventricle cavity were weighed to determine levels of hypertrophy. Transmural myocardial samples were dissected from the mid left ventricular free wall.

Snap frozen tissue samples and formalin fixed paraffin embedded (FFPE) sections were provided. FFPE sections were stained both with H&E (using the Ventana automated staining platform and reagents (Roche)) and Masson's Trichrome. In brief, FFPE sections were immersed in xylene (2×5 min), 100% ethanol, 90% ethanol, 70% ethanol, 50% ethanol (2 min each) followed by immersion for 60 min in preheated Bouin's solution (warmed to 60° C.) (HT10132, Sigma). Once cooled, slides were stained with Weigert's Iron Hematoxylin solution (HT1079, Sigma) for 5 min, rinsed with distilled water then stained with Biebrich Scarlet-Acid Fuchsin (HT151, Sigma) for 15 min. After rinsing in distilled water, slides were differentiated in Phosphotungstic/Phosphomolybdic acid solution (HT153, Sigma) for 10-15 min or until collagen was not red. Aniline Blue solution was applied to the slides for 30 min until collagen turned blue. Slides were rinsed in distilled water, then 1% acetic acid for 3 min. Slides were dehydrated in 2×90% alcohol and 2×100% alcohol followed by xylene. Slides were mounted with glass coverslips using Permount™ (Fisher Scientific).

### Western Blot Analysis of Human Heart Tissue

For isolation of protein from human heart tissue, less than 100 mg snap-frozen tissue was lysed in RIPA buffer supplemented with protease inhibitor cocktail (Millipore). Tissue was homogenised using a Polytron tissue homogeniser for 30 s on wet ice followed by centrifugation, for 15 min at 4° C., to pellet tissue debris. The supernatant was transferred to a fresh 1.5 ml tube and protein concentrations calculated. Lysates were subjected to SDS-PAGE and transferred to nitrocellulose membranes (Amersham Biosciences) for Western blotting. Blots were probed for β3-integrin (kind gift from Barry Coller, Rockefeller University), succinate dehydrogenase (Abcam, ab178423), pyruvate dehydrogenase (Abcam, ab 131263), aconitase (Abcam, ab129069) and GAPDH (Millipore, AB2302). Densitometric readings of band intensities were obtained using the JmageJ™ software.

### Immunofluorescence for β3-Integrin in Human Heart Tissue

Paraffin embedded tissues were dewaxed blocked in 10% normal goat serum, NGS, and 1% bovine serum albumin, BSA, for 1 h, followed by incubation with anti-β3 primary antibody (1:200) (Invitrogen) in blocking buffer (10% NGS, 1% BSA) overnight. Sections were incubated for 2 h at room temperature with secondary antibody in blocking buffer (10% NGS, 1% BSA) conjugated to 488 Alexa fluorophore, before being immersed in Sudan Black for 15 min to reduce autofluorescence. Sections were washed and finally mounted in prolong gold with DAPI. Stained sections were imaged on the Zeiss 710 confocal microscope and processed using Image J™ software.

### Abdominal Abdominal Aortic Constriction (AAC)

Abdominal AAC, a common experimental procedure, was used to elicit pressure overload-induced heart failure in murine models. AAC provides a time-dependent, reproducible model of HF characterised by a significant rise in left ventricular weight, deleterious myocardial dilation, and a reduction in cardiac inotropy, and rise in myocardial fibrosis. Physiologically this mimics some of the clinical features of human aortic stenosis.

Male C57BL6 mice (Charles River) underwent AAC surgery at 4-5 weeks of age (between 20 and 22 g). Mice were anaesthetised with isoflurane, placed in a supine position on the procedure table, and an incision made along the ventral underbelly, extending from the xiphoid process of the sternum, down to the groin to reveal the abdominal muscle. The abdominal muscle was then cut the same distance along the linea alba to allow easy reattachment of the two halves. The two sides of the abdominal muscles were clipped back to reveal the abdominal gastrointestinal organs, and these were carefully manoeuvred to expose the underlying abdominal aorta. The aorta was cleaned of visceral fats, and carefully separated from parallel vasculature, whilst avoiding any spinal nerves. The abdominal aorta was then constricted using a 6-0 silk suture tied to the width of a 27-gauge needle at a position rostral to the renal artery bifurcation. Great care was taken to ensure the knot remained taut after tying. This was to ensure minimal variability amongst aortic lumen occlusion between animals, and therefore minimise differences in the degree of aortic stenosis. The skin and abdominal muscles were closed with a 6-0 silk suture thread. SHAM operated mice underwent the same operation with the exception of aortic constriction. In an effort to ensure reproducibility amongst AAC surgeries only male mice weighing between 20 and 22 g (correlating to 4-5 weeks of age) were used. Echocardiography was performed up to 3 days prior to surgery, 3 weeks following surgery, and then at 6 (and/or 12 weeks) following surgery. At the experimental end-point, mice were sacrificed and their lungs, kidneys and hearts excised. Hearts were halved longitudinally and either snap frozen in liquid nitrogen (for immunohistochemistry) or stored in an RNA later stabilisation solution (for RT-quantitative PCR) (ThermoFisher Scientific, Hertfordshire, UK).

### Cilengitide Administration

Cilengitide (Bachem) was stored as a 2 M solution in saline at −20° C. prior to use, before thawing and serial diluting to an appropriate concentration with sterile saline for use. Injections were administered 3 times per week at the indicated doses intraperitoneally (IP). Low dose Cilengitide (ldCil) is classified as 50 μg/kg (27).

### Echocardiography

Cardiac function was assessed by transthoracic echocardiography using a VisualSonics Vevo 770 30-MHz transduction probe. Mice were anaesthetised with isoflurane (1.5% in O₂), hair removed from their chest with hair removal cream, and then were placed in a supine position onto a warmed pad (46° C.). The chest was covered in ultrasound gel, and the transduction probe was positioned adjacent to the mitral valve, at the tips of the papillary muscles, and images were taken just below this to ensure positional reproducibility. LV internal diameter (LVID) and LV posterior wall thicknesses at diastole (d) and systole (s) were measured from short-axis M-mode images. LV ejection fraction (EF %) was calculated as follows: LVEF %=[(LVIDd)³−(LVIDs)³]/(LVIDd)³×100; LV fractional shortening (FS %) was calculated as follows: LVFS %=(LVIDd−LVIDs)/LVIDd×100. Values were averaged across three beats. All efforts were made to ensure consistency between mouse echocardiographs, including: pad temperature to be maintained at 46° C., isoflurane anaesthesia at 1.5%, oxygen flow at 1.5 L/M, and cardiac function to be measured in the same anatomical location throughout.

### Mean Arterial Blood Pressure (MABP)

Blood pressure was recorded in anaesthetised mice via intra-arterial cannulation of a carotid artery 6 weeks post-AAC surgery, using a Microtip pressure catheter (Millar instruments). Mice were placed in a supine position on a pre-warmed heating mat (46° C.), and a midline incision made extending the length of the neck with surgical scissors. The large salivary glands were gently moved laterally, and a single carotid artery was located for cannulation. The carotid artery was blunt dissected to remove visceral fats, and then ligated with surgical suture at its most rostral and caudal ends, leaving a space of roughly 2 mm in between. A small incision was made between the two ties using fine dissection spring-tip scissors, and a microtip pressure catheter was inserted into the artery. The region of the artery housing the catheter was tied in place, and the caudal tie loosened to allow blood flow into the pressure catheter. Mice were allowed to recover for 5 min, or until the blood pressure trace steadied. MABP was then averaged across a 30 s period using MATLAB analysis. All efforts were made to ensure mouse body temperature, and levels of anaesthesia in particular, remained constant between animals.

### Immunofluorescence for Wheatgerm Agglutinin (WGA) and CD31 and Measurement of Cardiomyocyte Size and Blood Vessel Density

5 μm snap-frozen sections were fixed in 10% acetone, washed in phosphate buffered saline, PBS, blocked in 5% normal goat serum/PBS for 1 h at room temperature and incubated with WGA directly conjugated to Alexa Fluor 488 (Invitrogen) (1:100) in blocking buffer (5% normal goat serum/PBS) for 2 h at room temperature. Sections were washed in PBS and water then mounted in anti-fade gold mountant with DAPI (Molecular Probes, Invitrogen). Images were taken with a Zeiss Axioplan microscope, and the cardiomyocyte size was analysed with ImageJ™ software. 100-200 cells per heart were measured to determine cardiomyocyte size.

For anti-CD31 (BD Bioscience) detection, 5 μm snap-frozen sections were blocked in 5% normal goat serum in PBS for 1 h, followed by incubation with anti-CD31 primary antibody (clone H-3, sc-376764, Santa Cruz, 1:100) in blocking buffer (5% normal goat serum in PBS) for 2 h at room temperature. Next, sections were incubated for 1 h at room temperature with Alexa Fluor® 488-conjugated-secondary antibody (Invitrogen), before washing in PBS then water and mounted in anti-fade gold mountant with DAPI. Images were taken with a Zeiss Axioplan microscope connected to a digital camera. To quantify cardiac blood vessel density, the number of CD31-positive blood vessels present across an entire area of abdominally-orientated cardiac tissue was counted within a field of view taken at 40× magnification. 3 non-overlapping fields of view were used per heart and an average taken across fields.

### Cardiac Endothelial Cell Isolation

Primary mouse endothelial cells were isolated from the whole hearts of C57/BL6 3-7 day old mice. Hearts were removed, mechanically minced with scissors, and digested in pre-heated (37° C.) 10 ml 0.1% collagenase type I solution (Gibco Invitrogen) for 1 h. Usually 3 hearts were minced together for a single prep. The collagenase reaction was stopped by adding equal parts mouse lung endothelial cell (MLEC) media, and the digested tissue was homogenised in a dish by syringing up to 30 times with a 19 and 21-gauge needle, and then passed through a 70 μm pore-size cell strainer. Cells were centrifuged at 1500 rpm for 3 min, pellet was suspended and then incubated in a solution containing magnetic beads conjugated to a CD31 antibody (BD Pharmingen) to separate primary cardiac endothelial cells from the total cell population. Sorted cells were plated in T25 flasks coated with 0.1% gelatin, 10 μ/ml fibronectin and 30 μ/ml bovine collagen, suspended in mouse lung endothelial cell culture (MLEC) media and incubated at 37° C., 5% CO₂. Cells were washed 3 or 4 times in PBS the next day to remove excess red blood cells and other cell-debris and growing medium applied. Cardiac endothelial cells were split into new flasks when they reached 80% confluence. Mouse cardiac endothelial cells were split at a 1:2 ratio and were passaged no more than once.

### Cardiomyocyte Cell Isolation Culture

Primary mouse cardiomyocytes were isolated from the whole hearts of 1-3-day old C57/BL6 wild-type mice. Hearts were dissected and removed from the thoracic cavity, mechanically minced with scissors and digested in cardiomyocyte isolation enzyme 1 (200 μl per heart) and cardiomyocyte isolation enzyme 2 (10 μl per heart) (Pierce primary cardiomyocyte isolation kit, Thermo Scientific). Digested hearts were then homogenised by gently pipetting up to 30 times with a 1 ml pipette, and the resulting single cell suspension added to complete DMEM solution (DMEM, 10% FBS, 1% Penicillin Streptomycin stock solution). After combination of all cell suspensions, the total number of viable cells were counted, and plated at approximately 1 million cells per single well of a 6 well plate. The following day, wells were washed with PBS (without calcium and magnesium), and complete cardiomyocyte growth medium added. Complete cardiomyocyte medium thereafter was replaced every other day.

Following 5 days culture at 37° C., 5% CO₂, primary mouse cardiomyocytes were serum starved for 24 h in a 0.5 FBS-DMEM media. On day 6 cells were washed and refed with complete cardiomyocyte growth medium plus or minus 1 μM angiotensin II (Alfa Aesar), and this was repeated on day 7. In tandem to angiotensin II treatment, cells were either treated with vehicle or low dose Cilengitide (20 nM) (Bachem). On 8-day cardiomyocytes were lysed for RNA analysis.

### Western Blot Analysis of Cardiomyocytes

Cardiomyocytes were prepared and treated as above. At the end of the experiment, cells were lysed with RIPA buffer for 10 min on ice and the cell debris pelleted by centrifugation of the lysate at 9300 g for 10 min at 4° C. Protein concentration was determined from the cell supernatant and 20 μg protein loaded onto 10% gels. Membranes were probed for succinate dehydrogenase (Abcam, ab178423), pyruvate dehydrogenase (Abcam, ab131263), aconitase (Abcam, ab129069) and GAPDH (Millipore, AB2302). Densitometric readings of band intensities were obtained using the JmageJ™ software.

### Cardiomyocyte Size Assessment

For cardiomyocyte hypertrophy assessment, 1 μM angiotensin II (Alfa Aesar, ThermoFisher Scientific) was added to cardiomyocyte growth media post serum starvation (i.e. 6 days post-culture), and this was refreshed the next day. Images of cardiomyocytes were captured using a bright field microscope at 40× magnification at 0, 24, and 48 hours post-angiotensin II treatment and analysed using ImageJ™ software.

### RNA Analysis by RT-qPCR and RNA-Seq

Medium was removed and cells were washed 3 times in PBS. 350 μl RLT lysis buffer (Qiagen) (plus 1:100 beta-mercaptoethanol) was added to each well to disrupt and lyse the cells. Cell lysate samples were then combined with an equal volume of 70% ethanol, before RNA was extracted according to the manufacturer's instructions, before being stored at −80° C. until required. For whole hearts, less than 100 mg of tissue was lysed with 700 μl RLT lysis buffer (Qiagen) (supplemented 1:100 beta-mercaptoethanol) in a QIAshredder (Qiagen) for 10 min. Tissues were then processed as above with the RNeasy mini kit.

The purity and quantity of RNA was analysed using a Spectophotometer, (NanoDrop, Wilmington, US). All samples were expected to exhibit a 260/280 ratio of approximately 2.0, indicating that the RNA was pure and free of nucleic acid contaminants, and a 260/230 ratio of >1.8, which was used as a secondary measure of nucleic acid purity. For RNA-Seq, RNA integrity (RIN) was assessed using gel electrophoresis to evaluate the ratio of 28S to 18S rRNA.

Complementary DNA (cDNA) synthesis was carried out using the Applied Biosystems High Capacity cDNA Reverse Transcription (RT) Kit on a thermal cycler as per the manufacturer's instructions. cDNA was made up to a total quantity of either 600 (most mouse tissue and/or cells), or 1000 ng (human heart tissue).

Quantitative PCR (qPCR)

Analyses were performed using the ABI Prism 7500 Sequence Detection System Instrument and software (PE Applied Biosystems). qRT-PCR was performed using sample cDNA (FAM-tagged), an internal control GAPDH (VIC-tagged) and specific TaqMan probes. qPCR was carried out using the TaqMan Universal PCR Master Mix (PE Applied Biosystems) in a 96 well plate. 160 ng of cDNA from each sample was amplified using qPCR across 40 cycles. Target mRNA was normalised to GAPDH (HPRT-1 if human), and the expression level of each gene determined relative to the initial experimental controls using the 2^{-ΔΔCT} method.

### Example 1: RNA-Seq Analyses and Comparison with Human Heart Data

RNA-Seq was performed by Barts and the London Genome Centre on the Illumina NextSeq 500 platform, generating on average ˜15-17 million paired-end reads of 76 bp in length per sample. After quality check using FastQC, raw reads were mapped to the mouse genome (mm10, Genome Reference Consortium GRCm38) in the strand-specific mode using HISAT2 (65). Number of uniquely aligned reads (q>10) to the exonic region of each gene were counted using HTSeq (66) based on the GenCode annotation version M16 (Ensembl v91). Only genes that achieved at least one read count per million reads (cpm) in at least four samples (i.e., number of samples per group) were kept. Conditional quantile normalisation (cqn) was then performed accounting for gene length and GC content, and a log₂ transformed reads per kilobase of transcript per million mapped reads RPKM expression matrix was subsequently generated. Differential expression analysis was performed using the ‘limma’ R package (67). Gene-set enrichment analysis (GSEA) was performed using the GSEA software (68) to identify dysregulated canonical pathways curated in the Molecular Signatures Database (MSigDB-C2-CP v6.2) (FDR q<0.1). RNA-Seq data have been deposited in Gene Expression Omnibus (GEO) under the accession number GSE127854.

The GSE57338 microarray dataset of a total of 313 individuals which included 95 ischemic patients, 82 dilated cardiomyopathy patients, and 136 individuals with non-failing hearts, was downloaded from GEO. Differential expression analysis was performed using the ‘limma’ R package comparing ischemic vs non-failing and dilated vs non-failing. GSEA was performed using the GSEA software (PMID:16199517) to identify the dysregulated canonical pathways as described above. The overlap of differentially expressed gene and pathways (p<0.05) was then examined in relation to the mouse cardiomyocyte data. All graphics and statistical analyses were performed in the statistical programming language R (version 3.1.3).

### Example 2: β3-Integrin Expression is Elevated in Human Dilated and Ischaemic Cardiomyopathies

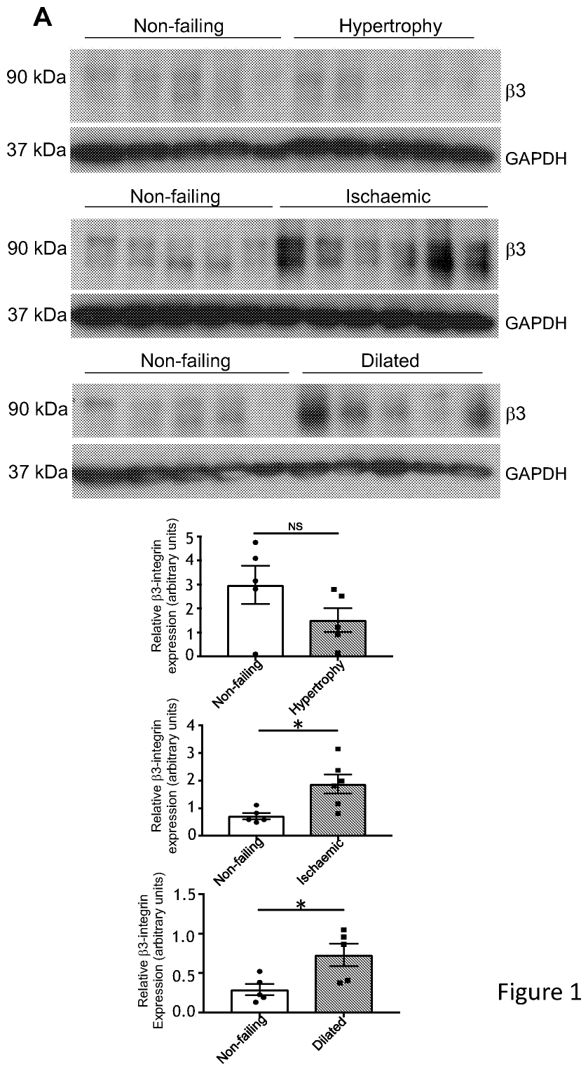

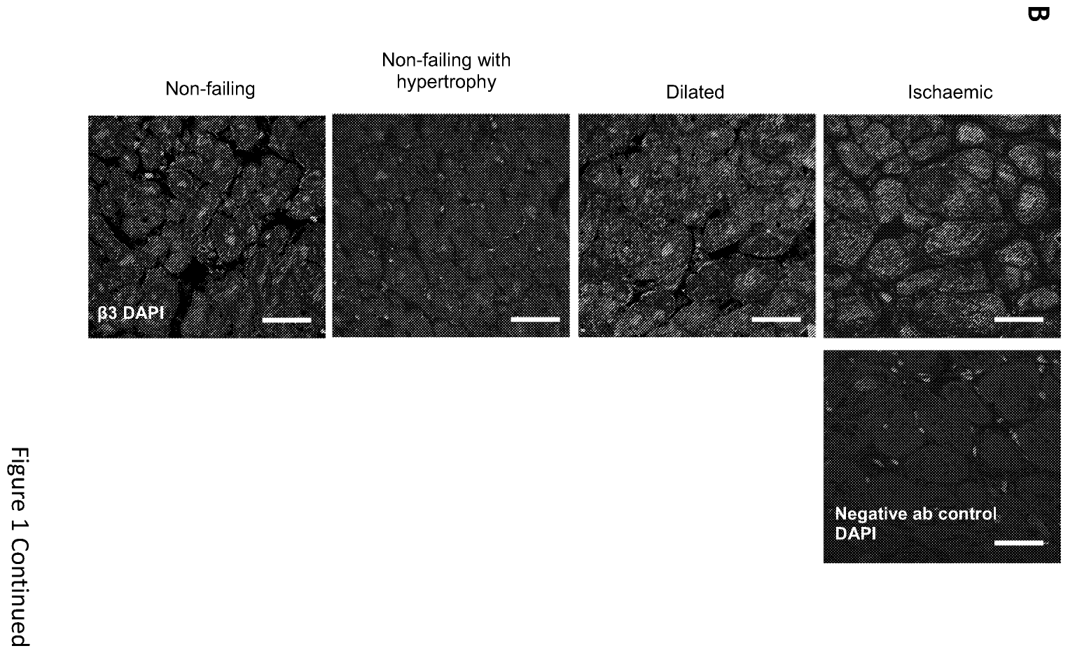

To test the utility of a low dose of Cilengitide, ldCil, in the treatment heart failure, the expression of its target, β3-integrin, in human non-failing, non-failing hypertrophic, dilated and ischaemic cardiomyopathies was examined. Western blot analysis for β3-integrin expression in human left ventricular biopsy lysates from separate patients indicated that although there were no differences in β3-integrin levels between non-failing and non-failing hypertrophic hearts though significantly elevated levels of β3-integrin were observed in both human dilated and ischaemic cardiomyopathies when compared with non-failing heart tissue (FIG. 1A). Immunostaining for β3-integrin showed that β3-integrin is present in human non-failing, dilated and ischaemic hearts but less so in non-failing with hypertrophy corroborating western blot results (FIG. 1B).

Together these data suggest that β3-integrin is apparent in human heart failure tissue and thus could provide a possible target for treatment. The pathological features of the human tissue were identified by H&E and Masson' s Trichrome staining of the FFPE sections (FIG. 6).

### Example 3: Experimental Heart Failure

Abdominal aortic constriction (AAC) surgery in mice recapitulates the salient features of human heart failure including pressure overload-induced left ventricular cardiac hypertrophy and fibrosis (29, 30) and provides a model to test new therapeutic strategies for the treatment of this condition. eIt was hypothesised that ldCil treatment may enhance cardiac angiogenesis and blood flow after AAC surgery and thereby provide a strategy to correct the pathobiology of heart failure.

Cardiac function was quantified by fractional shortening (FS %) first in a prevention mode experimental trial. Cilengitide, or vehicle alone as a control, was administered to mice 3 times a week for 7 weeks immediately after SHAM or AAC surgery. At the experimental endpoint (7 weeks post-surgery) mice that had undergone AAC surgery and treated with vehicle alone showed significantly reduced fractional shortening compared with mice that had SHAM surgery with vehicle treatment.

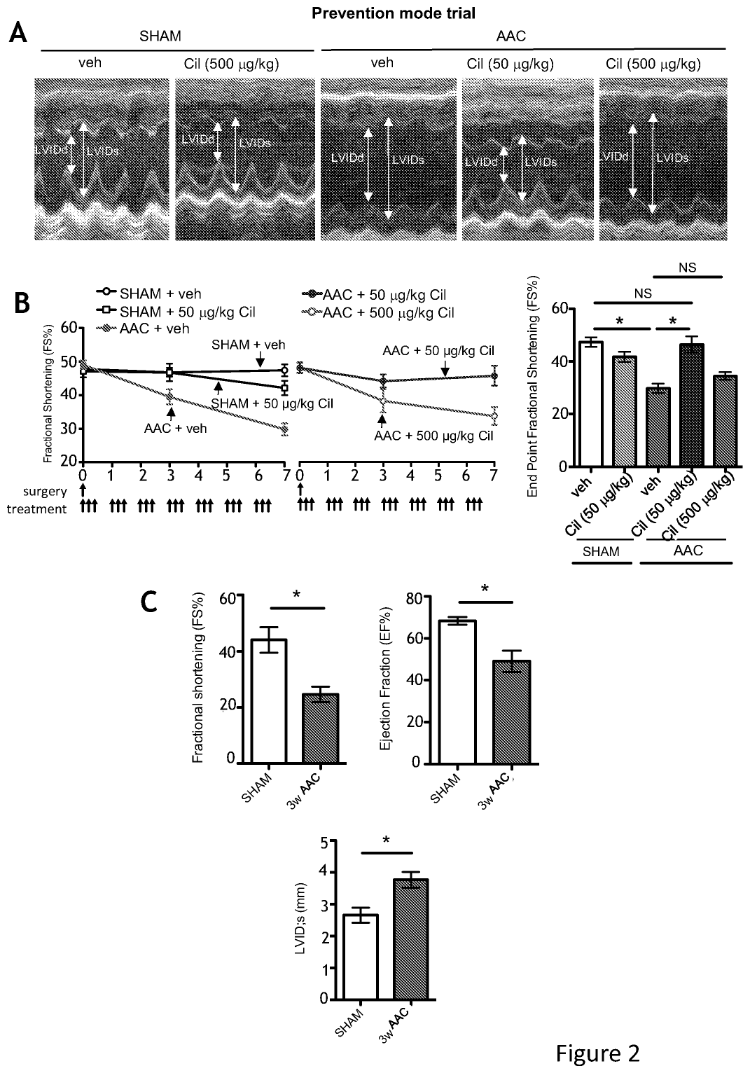

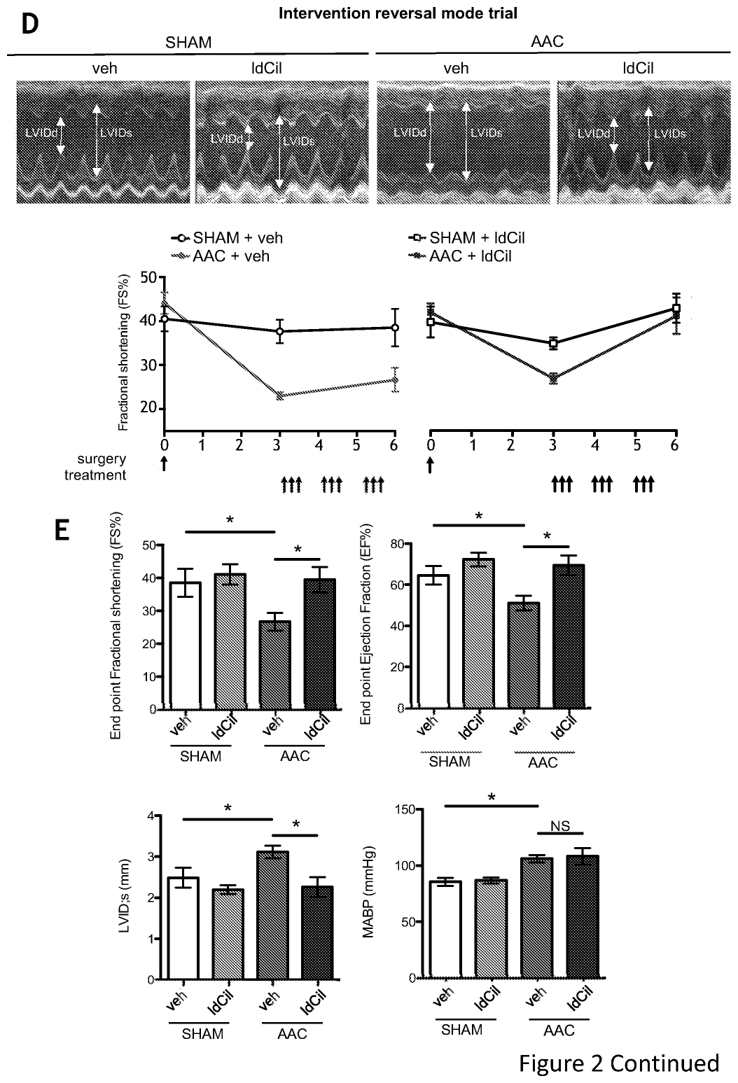

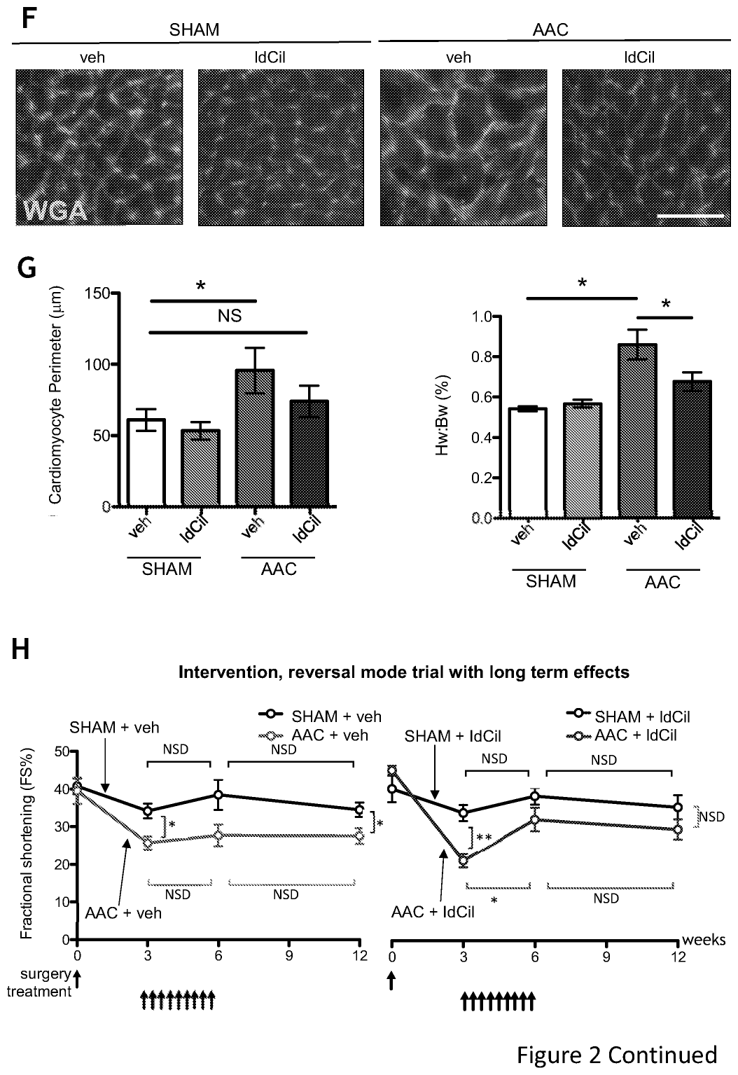

Administration of ldCil (50 μg/kg) after SHAM surgery also had no significant effect on fractional shortening. In contrast, treatment of mice with ldCil (50 pg/kg) but not a higher dose of 500 μg/kg, rescued the effect of AAC surgery and restored fractional shortening to that observed in SHAM treated mice (FIG. 2A, B). These data suggested that treatment of mice immediately after AAC surgery with ldCil was sufficient to rescue adverse heart failure effects in fractional shortening.

The effect of ldCil was also tested in a more clinically-relevant intervention reversal study starting treatment three weeks after AAC surgery i.e., after heart failure symptoms were established. Three weeks post AAC surgery FS %, and ejection fraction (EF %) were significantly reduced while left ventricle systole (LVID) was increased when compared with mice that had undergone SHAM surgery (FIG. 2C). To test the effect of ldCil treatment, mice underwent either SHAM or AAC surgery and three 3 weeks later were treated with either vehicle or ldCil 3 times a week for 3 weeks. Results indicate that at the end of treatment, although vehicle treatment had no apparent modifying effects, ldCil treatment rescued the effects on endpoint FS %, EF %, and LVID (FIG. 2D, E). Importantly, these effects were independent of changes in mean arterial blood pressure (MABP) (FIG. 2E). In addition to these functional features mice that have undergone AAC surgery typically exhibit adverse effects of enhanced cardiac hypertrophy with concomitant increased heart:body weight ratios (29, 31). Wheat germ agglutinin (WGA), binds cell membrane glycoproteins, and is used to determine cross-sectional sizes of myocytes as a measure of cardiomyocyte hypertrophy. Cardiomyocyte perimeter measurements in WGA stained sections of heart from experimental end points were reduced in ldCil treated mice after AAC surgery compared with vehicle (FIG. 2F). Further, ldCil treatment blunted increases in heart:body weight ratios in mice after AAC surgery (FIG. 2G). Thus, in this intervention mode study, ldCil treatment restores cardiac function and structure in the AAC model of heart failure to levels observed with SHAM surgery.

To test whether ldCil treatment efficacy was maintained, even after treatment cessation, mice were given ldCil or vehicle from 3-6 weeks post SHAM or AAC surgery and the effects on cardiac contractility assessed up to 12 weeks post-surgery. Results indicated that FS % was not significantly altered between 3 and 6 weeks or 6 and 12 weeks post SHAM surgery in either vehicle-alone or ldCil treated mice. As expected, fractional shortening was significantly reduced in vehicle -treated mice after AAC surgery. This reduction in FS % was sustained up until the 12-week endpoint of the experiment, indicating the persistent long-term effects of AAC surgery in vehicle-alone treated mice. In contrast, treatment with ldCil elevated fractional shortening significantly between 3 and 6 weeks after AAC surgery. This phenotypic rescue was sustained up until 12 weeks post-surgery at which point there was no significant difference between ldCil treatment that had undergone SHAM or AAC surgery (FIG. 2H). These data indicate that intervention treatment with ldCil is sustained after cessation of treatment, is disease modifying and potentially provides an advantage over current treatment strategies.

Example 4: Treatment with low dose Cilengitide enhances cardiac angiogenesis in vivo

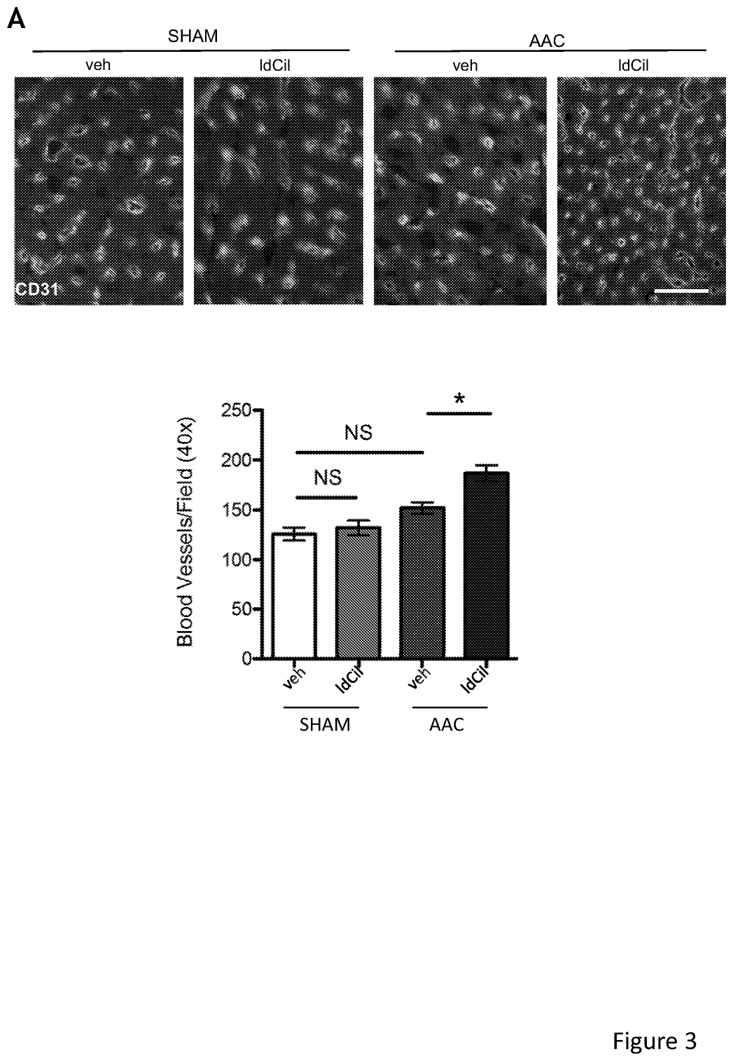

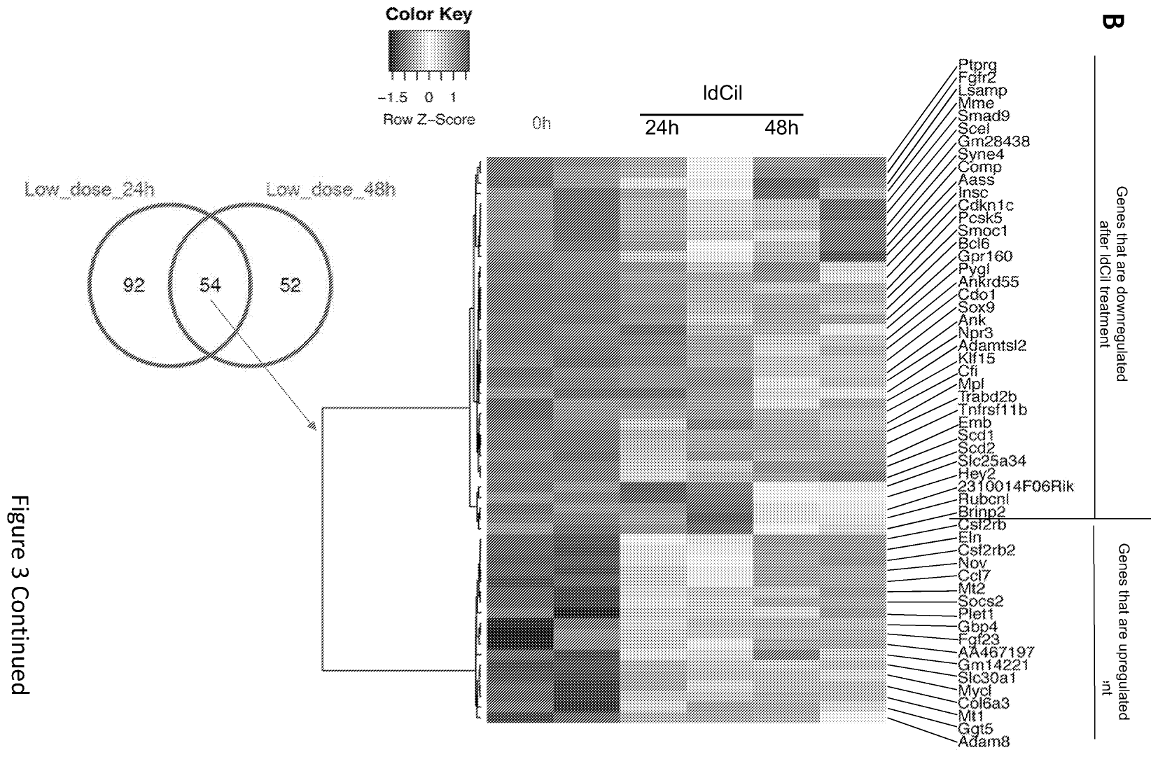

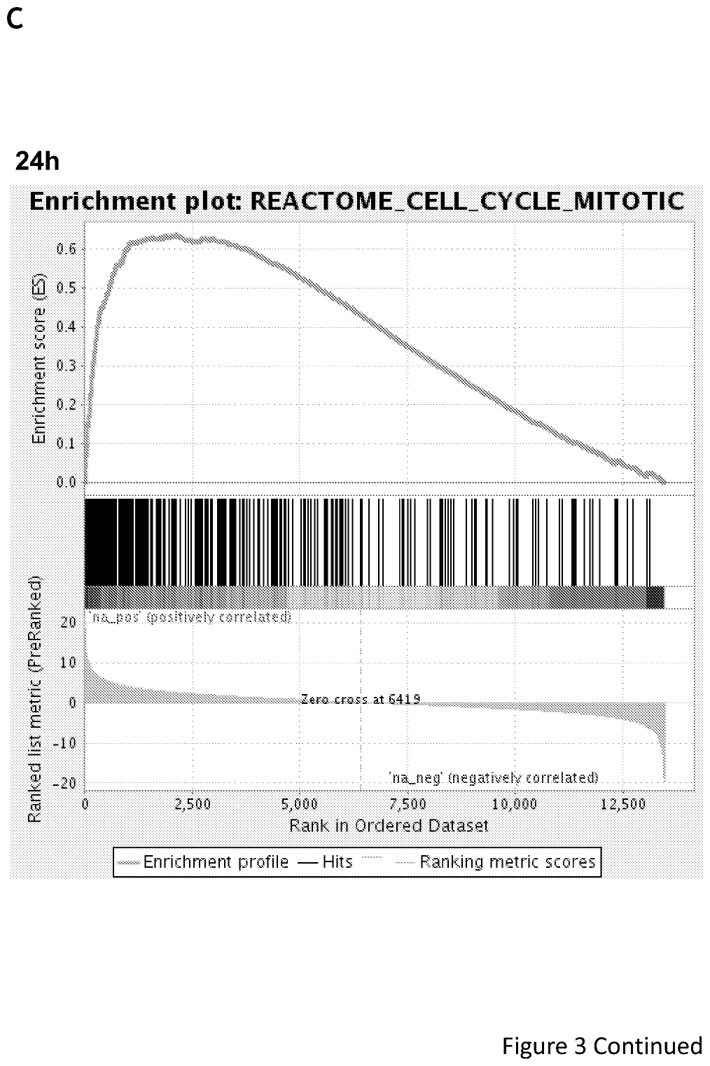

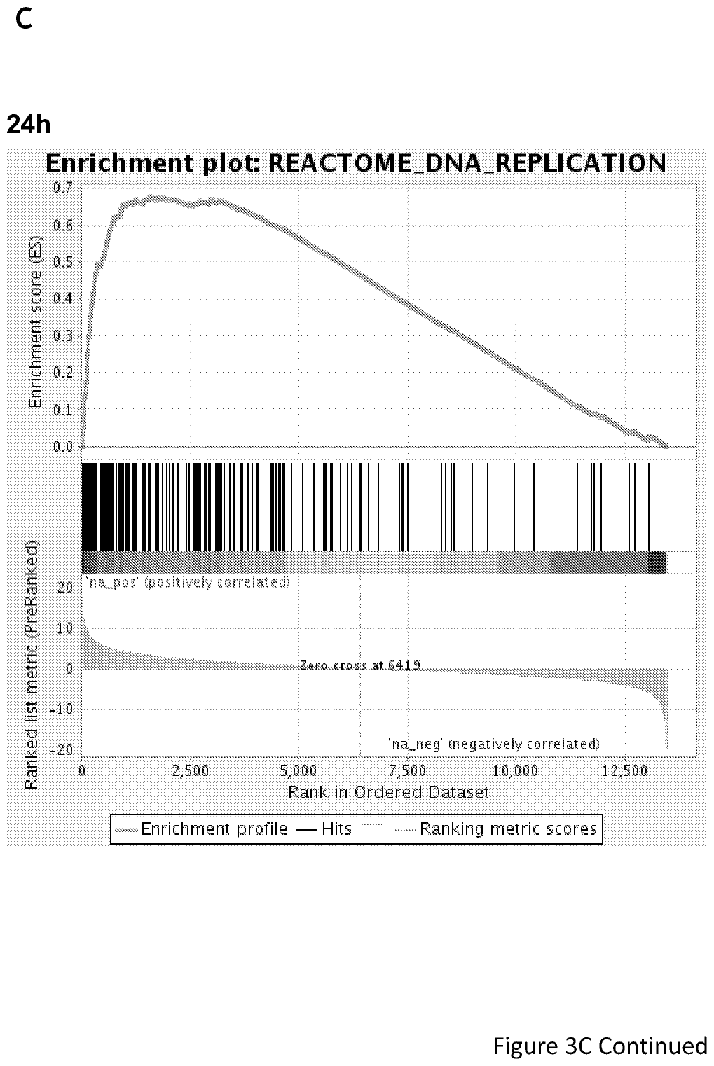

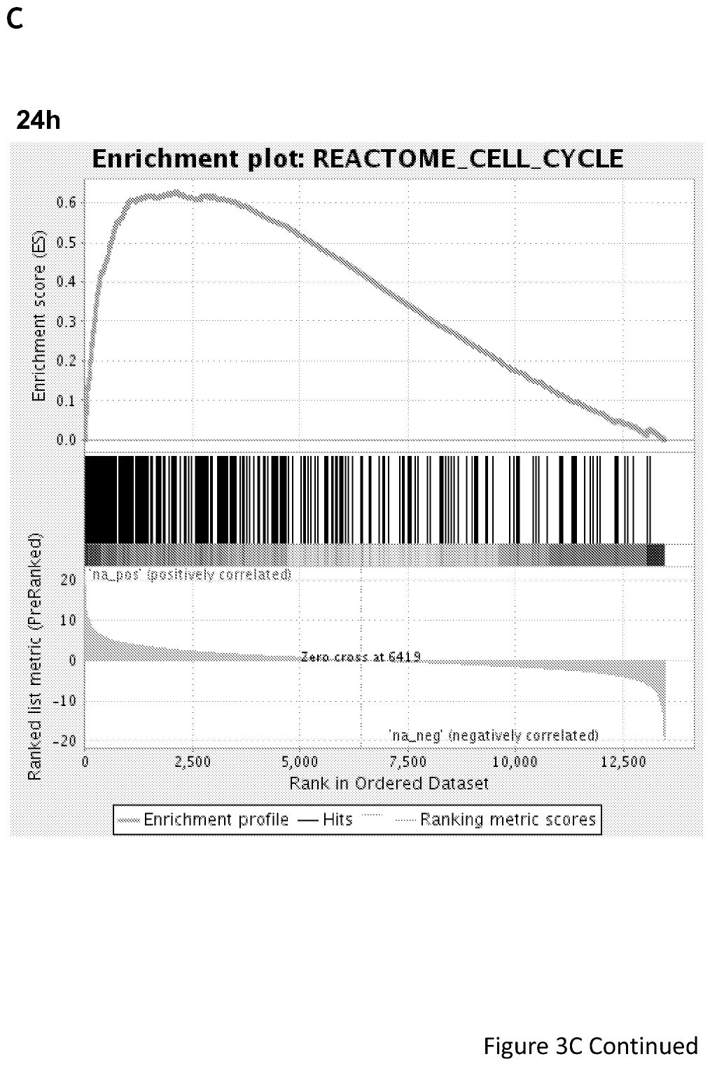

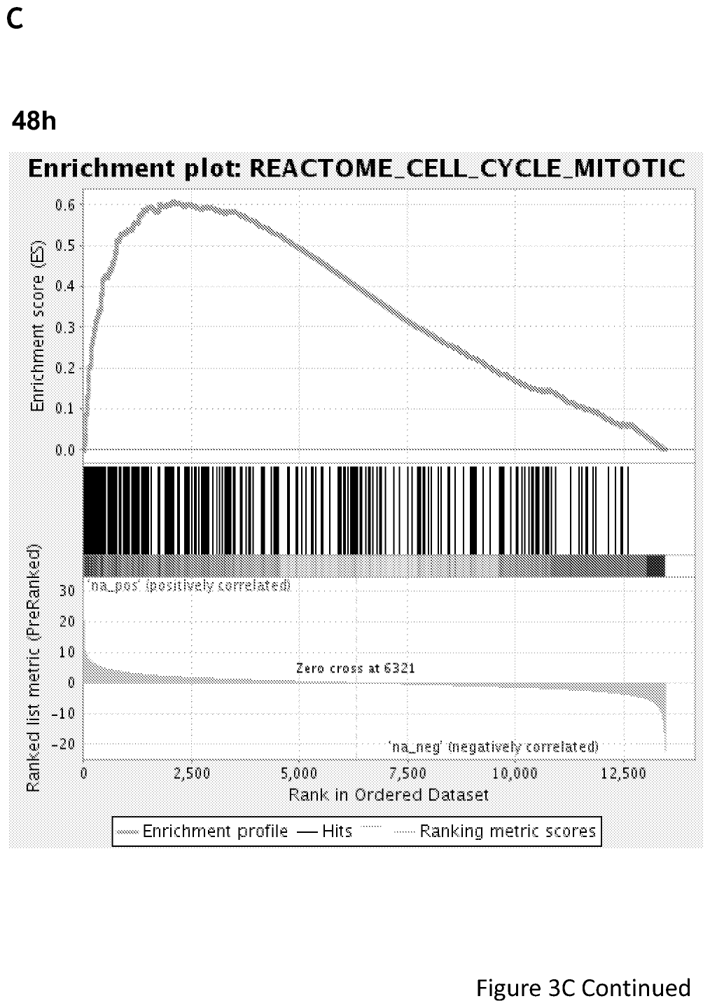

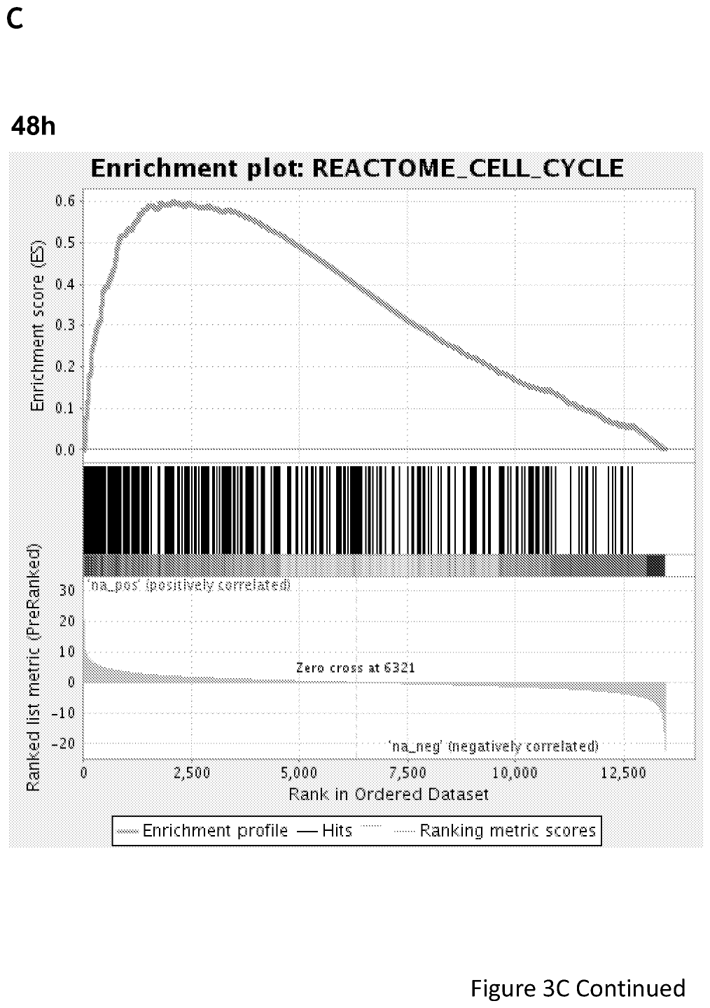

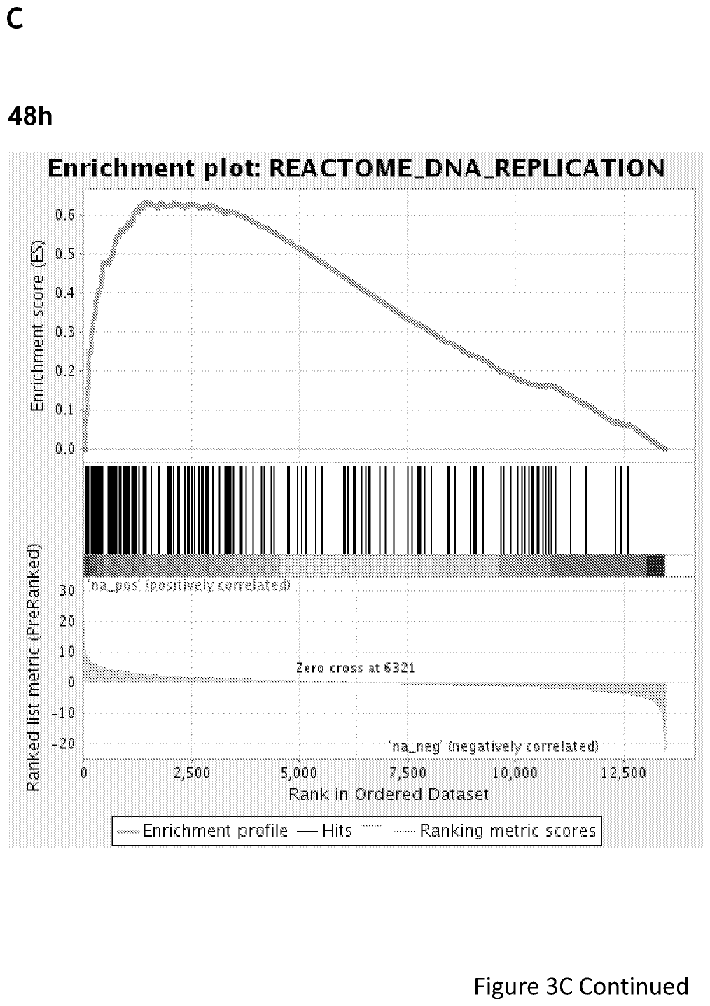

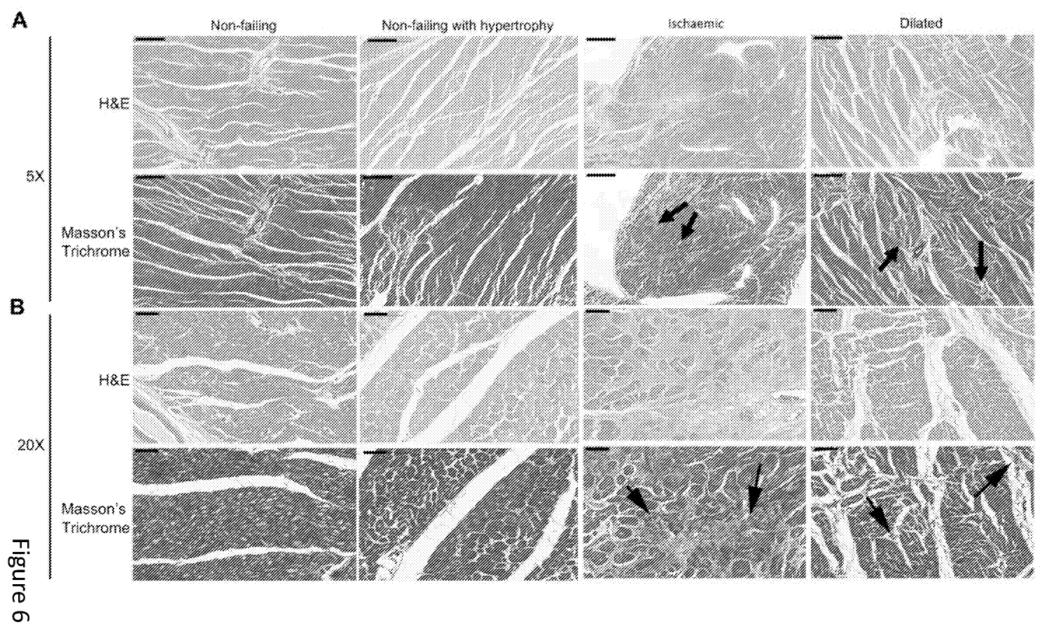

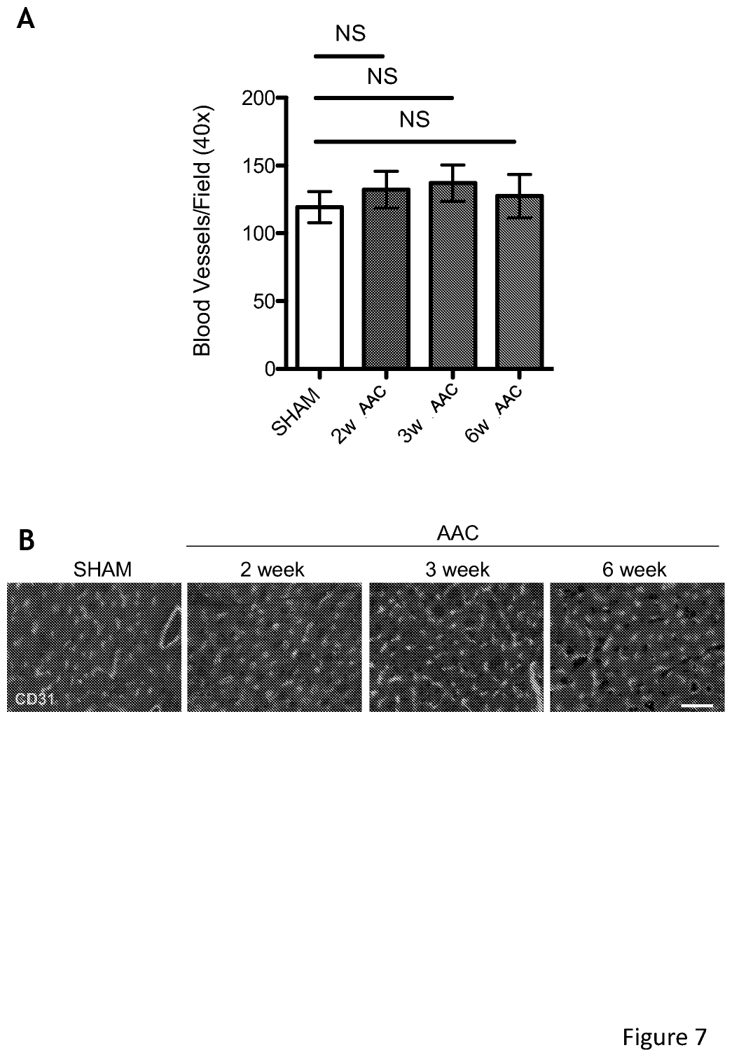

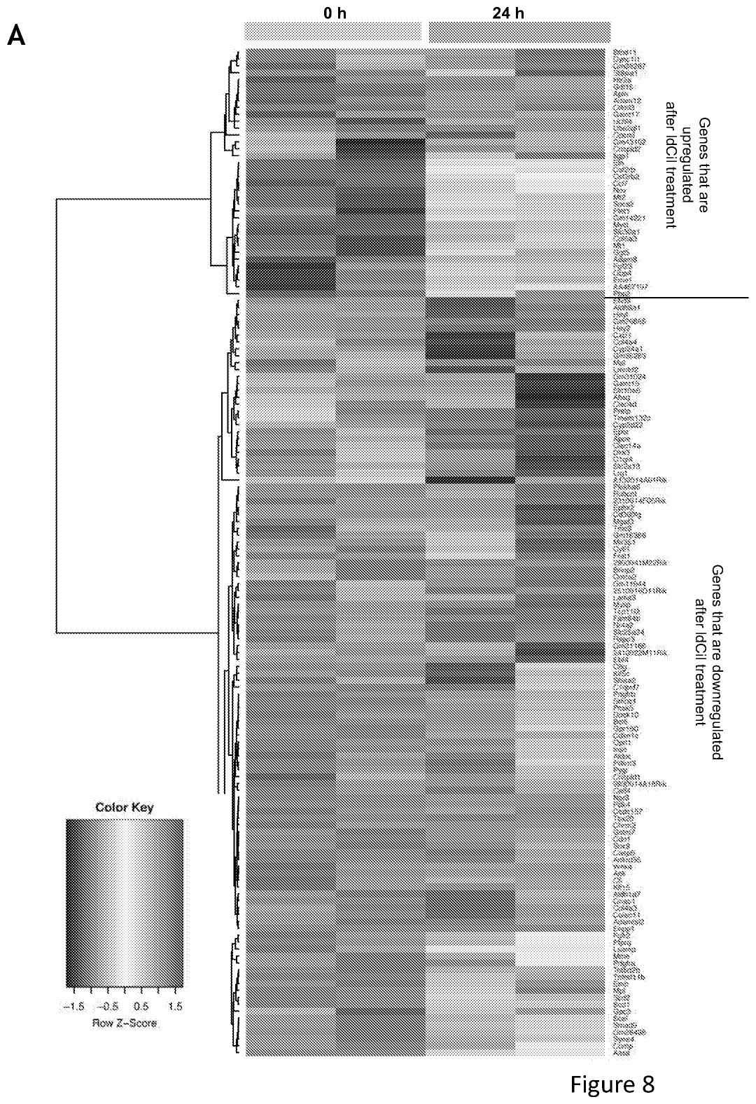

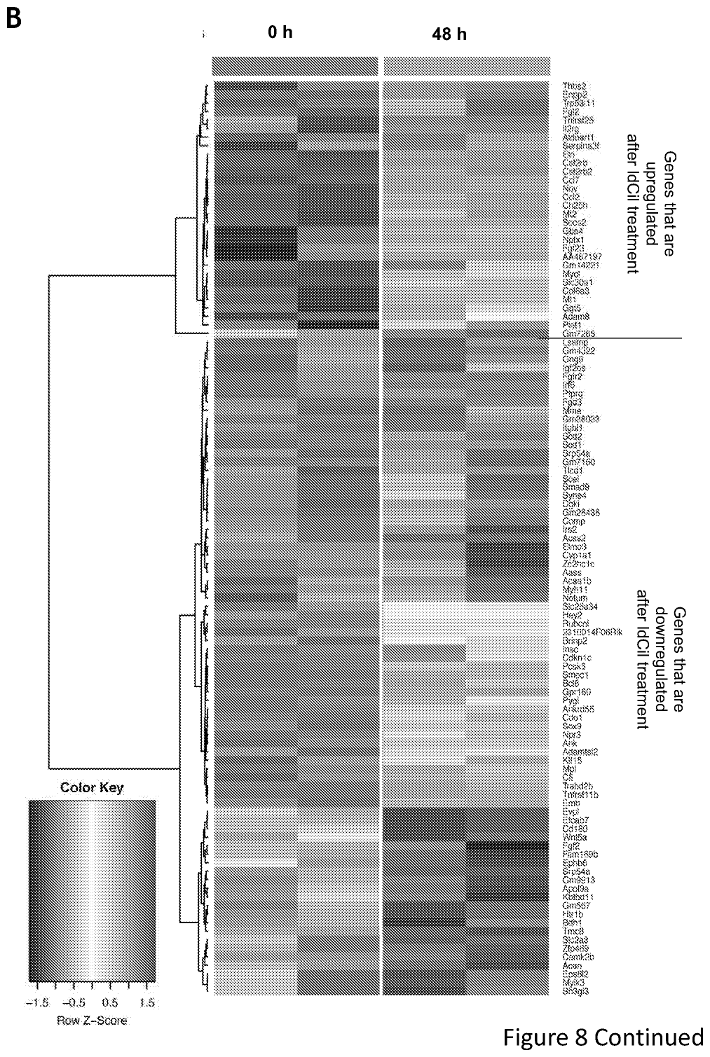

The physiological and molecular changes associated with the effect of ldCil in the AAC model were examined. Since heart failure is associated with reduced blood flow to the heart, cardiac capillary density after AAC surgery was examined. At 2, 3, and 6 weeks post AAC surgery heart sections were immunostained with an anti-CD31 antibody to detect blood vessels and the number of blood vessels per field of view was examined. Results showed that capillary density was not affected in the heart after AAC (FIG. 7). In contrast, ldCil treatment significantly enhanced cardiac blood vessel density in mice after AAC surgery but had no significant effect in mice that had undergone SHAM surgery (FIG. 3A). . The data indicates that the elevated cardiac function induced by treatment with ldCil after AAC surgery is associated with enhanced cardiac blood vessel density. At the molecular level RNA-Seq analysis was carried out to define transcriptomic profiles of mouse cardiac endothelial cells treated with ldCil for 24 and 48 hours. Results identified 54 concordantly differentially expressed genes at 24 h and 48 h ldCil treatment (FIGS. 3B and 8). Indeed, common differentially upregulated genes at both 24 h and 48 h with ldCil treatment included several known or putative proangiogenic or cardioprotective regulators such as: Csf2rb (32); Eln (33); Nov (a known ligand of αvβ33 integrin (34)); CCL7 (known to improve cardiac repair (35)); Mt2 (36); Socs2 (37); Fgf23 (known to protect against cardiac dysfunction (38)); Slc30a1 (39); Col6a3 (40); Mt1 (41); Ggt5 (42) and Adam8 (43). Furthermore, enrichment analysis of RNA-Seq data, at both timepoints, showed significant increases in gene sets involved in mitosis, DNA repair and cell cycle; all features of enhanced angiogenesis and the myocardial response to injury/pressure overload (FIG. 3C).

### Example 5: ldCil Treatment Restores the Transcriptomic Profiles in AngII-stimulated Model of Cardiac Hypertrophy Similar to Those Found in Non-Failing Human Heart

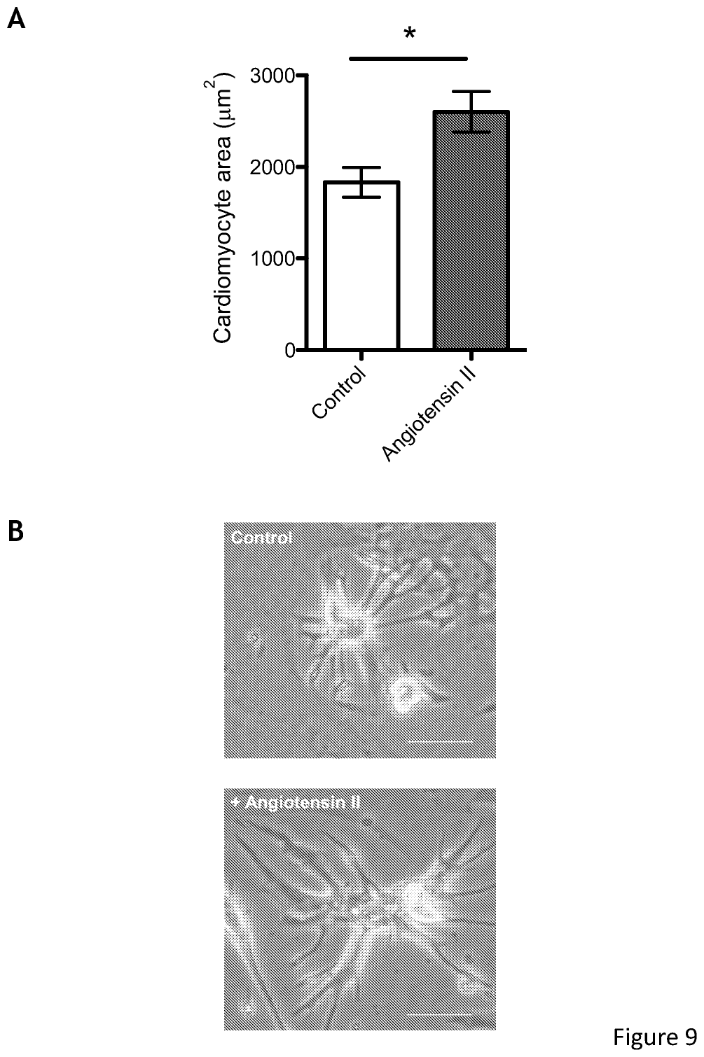

In addition to the effect of ldCil on endothelial cells, the direct effect of ldCil on cardiomyocytes was examined. Previous studies have identified that angiotensin II (AngII) treatment of cardiomyocytes mimics many features of hypertrophy observed in heart failure in vivo (44). AngII-stimulated cardiomyocyte cultures exhibit a significant increase in cell size indicative of hypertrophy such that AngII-stimulation of cardiomyocytes can be used as a model for hypertrophy (FIG. 9) (44).

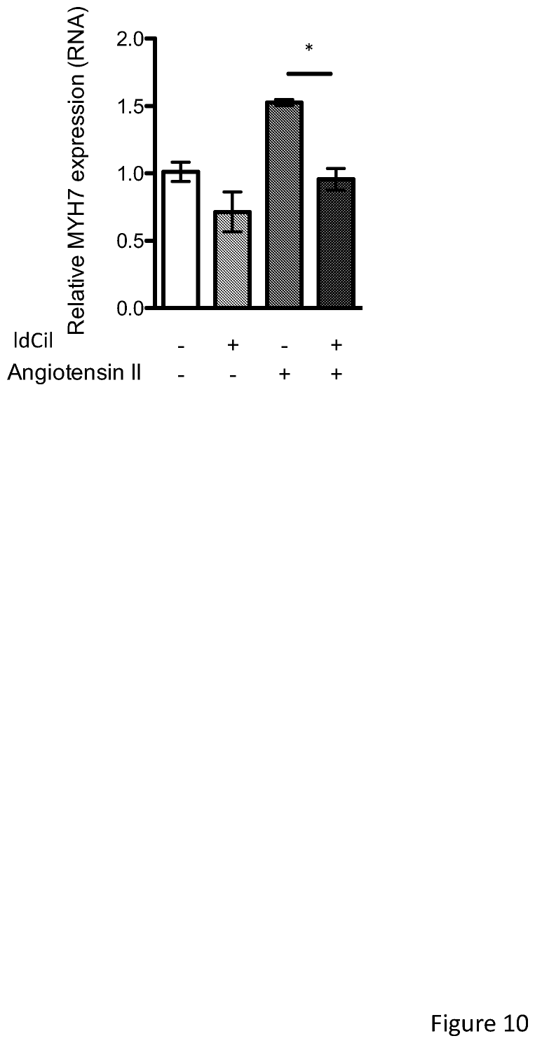

Another key feature of hypertrophy observed in both human heart failure and AngII treated mouse cardiomyocytes is the enrichment of foetal-like gene programmes involved in cardiac remodelling (45), including upregulation of hypertrophic markers such as Myh7. Importantly, additional treatment with ldCil restored Myh7 levels back to untreated levels (FIG. 10). These data not only validated the utility of AngII-stimulation of cardiomyocytes as a model for hypertrophy, but also indicated that ldCil has a direct effect on cardiomyocytes to restore this feature of hypertrophy.

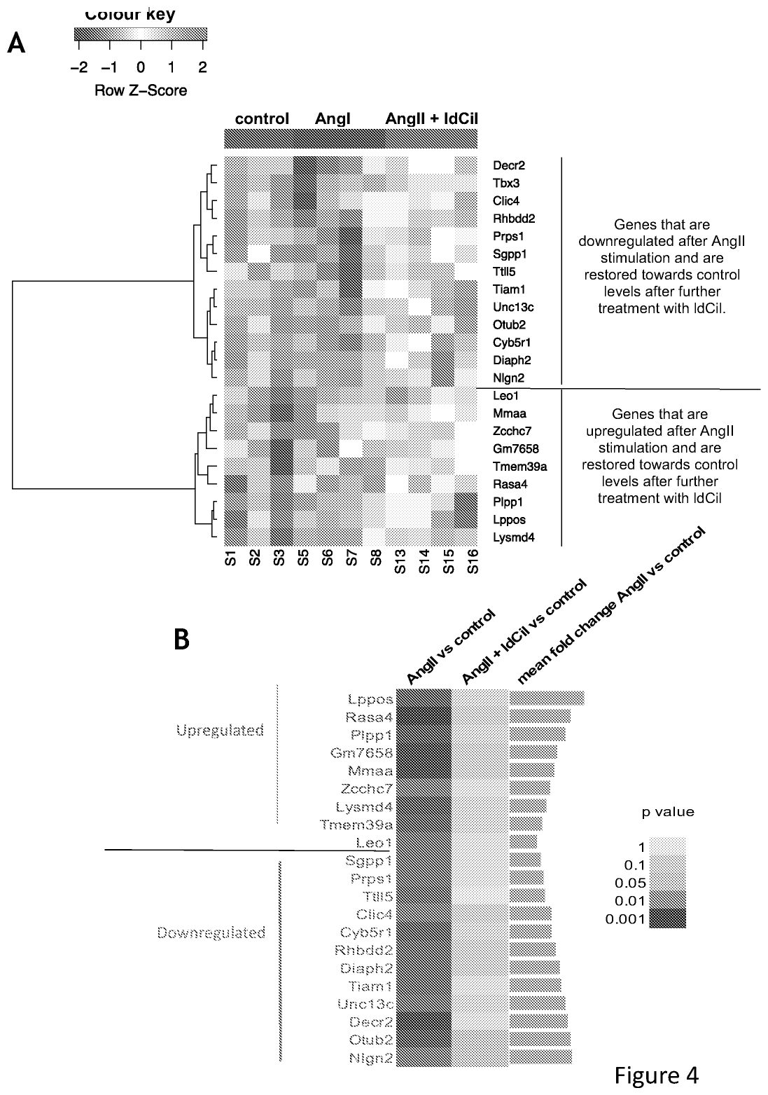

Additionally, RNA-Seq transcriptomic analysis demonstrated that AngII-stimulation of cardiomyocytes resulted in significant differential expression changes in multiple genes (FIG. 4A, B). Some of the most significantly differentially expressed genes included the upregulation of LIM domain containing preferred translocation partner (Lppos) the haploinsufficiency of which has been published to be involved in developmental heart defects (46); Rasa4, RAS p21 protein activator1 which has been shown to regulate cardiac fibroblast activation, although no known function in cardiomyocytes has been reported to date (47), and the downregulation of neuroglin 2, Nlgn2, polymorphisms which have been shown to be associated with blood pressure changes (48), whilst Otub2, that encodes the ovarian tumour domain (OTU)-containing subfamily of deubiquitinating enzymes has been associated with multisystemic human disorder including heart failure (49).

Treatment with ldCil restored the statistically significant transcriptomic alterations induced by AngII back to control treated cardiomyocyte levels for all candidates shown (FIG. 4A, B). Gene sequence enrichment analysis showed that AngII stimulation induced significant differential expression of multiple pathways (FDR q<0.1). Once again, after additional treatment with ldCil, these changes were no longer significant (FDR q>0.1) (FIG. 4C) suggesting that treatment with ldCil rescues the signalling pathway alterations induced by AngII. In particular the significant increase in TCA and oxidative stress pathways associated with AngII exposure and associated with some forms of heart failure (50) are restored by treatment with ldCil.

Additionally, PI3K and Akt signalling pathways are most significantly downregulated after AngII exposure and are known to be involved in heart failure responses (51). Indeed, the fold change restoration of both these pathways are most highly expressed after treatment with ldCil (FIG. 4C). Together, these data indicate that the direct effects of ldCil on cardiomyocytes are likely involved in restoring the pathobiology and molecular effects of hypertrophy.

### Example 6: ldCil Treatment Restores Transcriptomic Changes Associated with Non-Failure in Human Heart

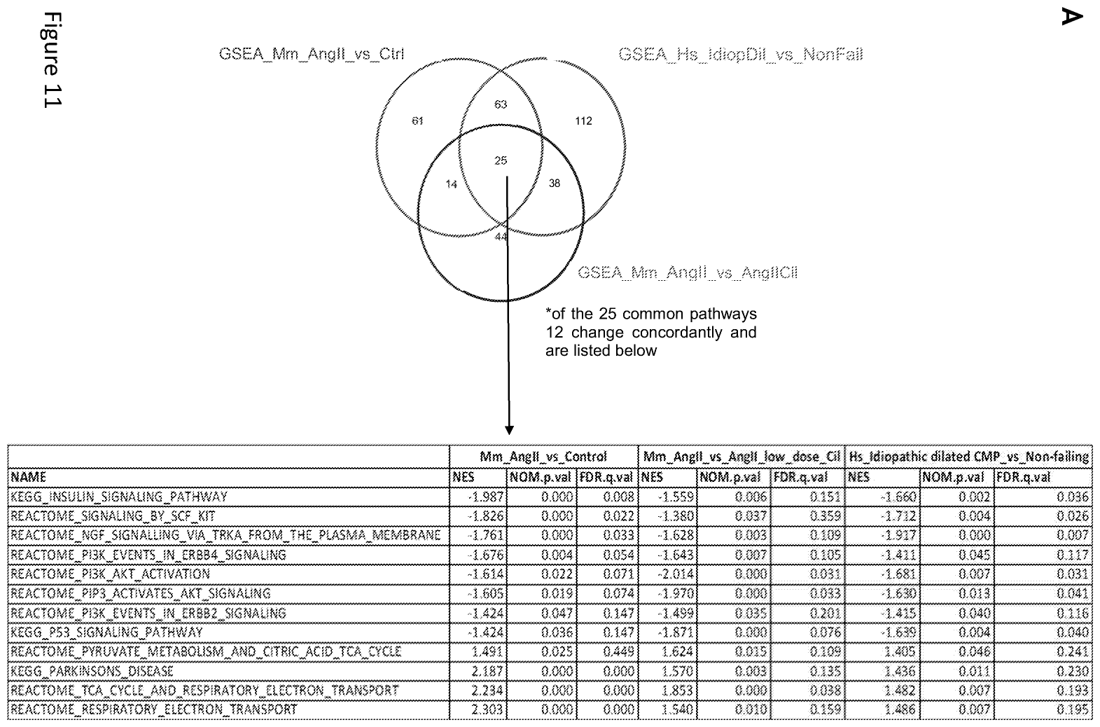

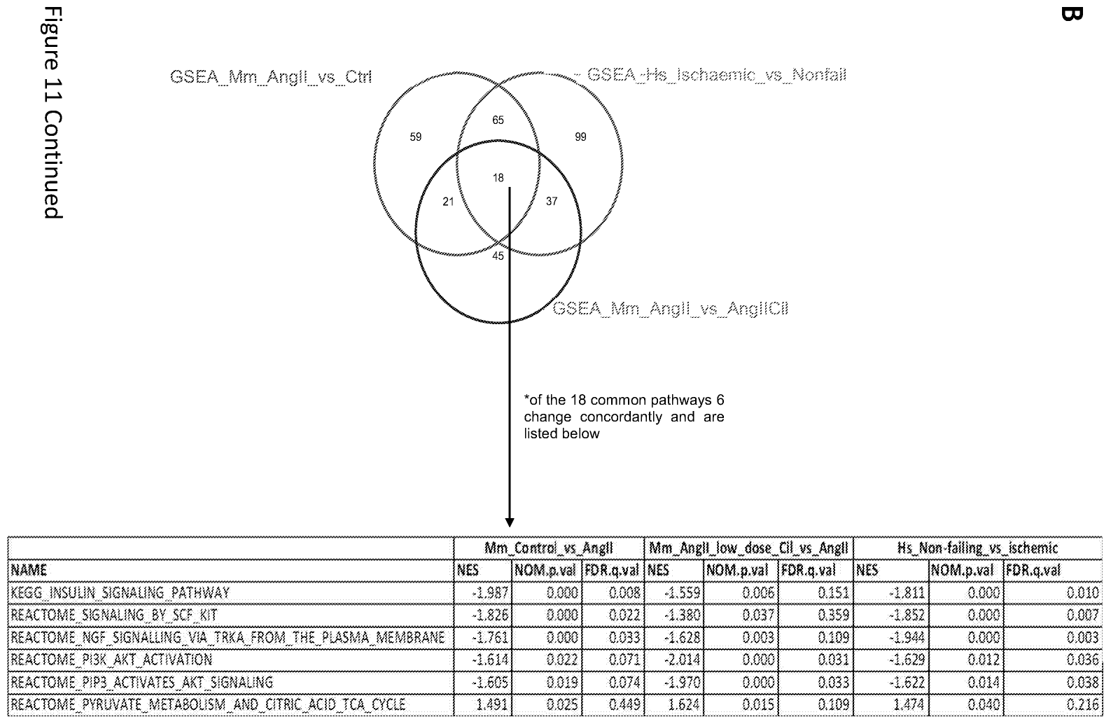

Previous work, using RNA isolated from bulk human cardiac samples, has identified the transcriptomic changes that are significantly regulated in human heart failure (52). To examine the relevance of findings in mouse tissue to human heart disease, we identified concordant transcriptomic changes published for human failing heart (ischemic and/or dilated vs human non-failing) vs those in the mouse model of hypertrophy (AngII-stimulated cardiomyocytes vs control treated cardiomyocytes). We found 290 orthologous genes altered concordantly in mouse cardiomyocytes and human failing vs non-failing heart. A subgroup of these genes—13 downregulated and 8 upregulated—were restored to non-failing human heart levels after treatment of mice with ldCil (FIG. 5A). Furthermore, out of the 163 differentially regulated pathways in AngII-stimulated mouse cardiomyocytes vs control cardiomyocytes, 88 were common with the 238 differentially enriched pathways in human dilated heart vs non-failing heart (FIGS. 5B and 11A). GSEA analyses revealed that 4 concordantly upregulated and 8 downregulated pathways in human idiopathic dilated cardiomyopathy vs non-failing heart and mouse AngII-stimulated cardiomyocytes vs control were restored to non-failing human heart profiles after ldCil treatment (FIG. 5B heatmap and FIG. 11A). Similarly, out of the 163 differentially regulated pathways in AngII-stimulated mouse cardiomyocytes vs control cardiomyocytes, 83 were common with the 219 differentially enriched pathways in human ischemic heart vs non-failing heart (FIGS. 5C and 11B). GSEA uncovered that 6 concordant pathways, 1 upregulated and 5 downregulated, in human ischemic heart vs non-failing heart and mouse AngII-stimulated cardiomyocytes vs control, were restored after treatment with ldCil (FIG. 5C, heatmap and FIG. 11B). Additionally, some of these concordantly enriched pathways were similar for both idiopathic dilated and ischemic cardiomyopathy including insulin signalling pathways, SCF-Kit signalling, NGF signalling, PI3K signalling and AKT signalling suggesting common mechanistic signatures that are relevant to the recovery of both cardiomyopathies.

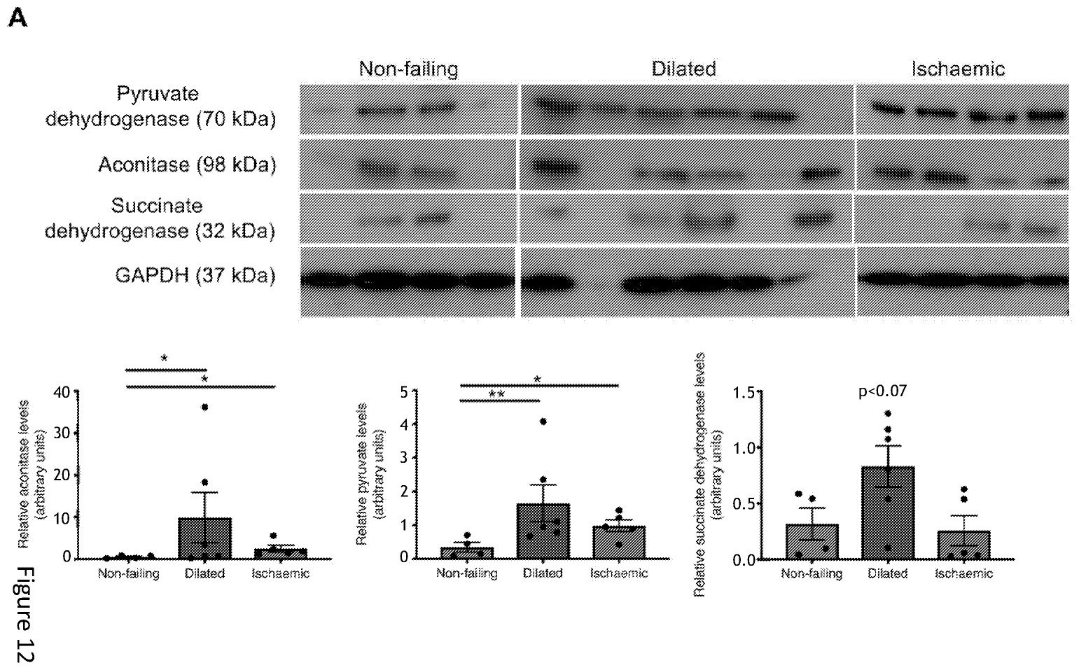

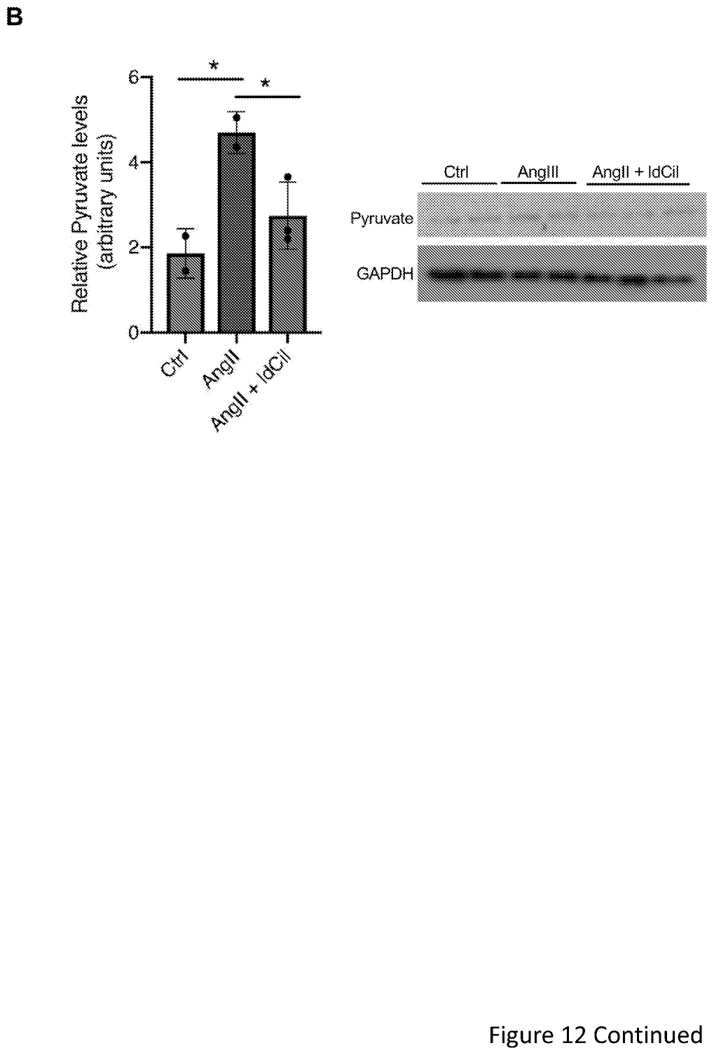

Western blot analysis validated some of the differentially expressed TCA-cycle intermediate players at the protein level (FIG. 12). Together, these data suggested that treatment of AngII-stimulated cardiomyocytes with ldCil reveals molecular profiles that are associated with human non-failing heart.

### Discussion

Heart failure is a common feature of the final stages of several cardiovascular diseases (e.g. hypertension, MI) with characteristic pathologies of reduced microvascular blood flow and cardiomyocyte hypertrophy. Importantly, disease modifying strategies with longer lasting pharmacodynamics are required.

The data disclosed herein shows that treatment with ldCil can enhance cardiac angiogenesis in the AAC model of heart failure and inhibit pathologic hypertrophy in cardiomyocytes. Moreover, ldCil treatment recapitulates some of the transcriptomic profiles of human non-failing hearts suggesting that this strategy may have relevance to human heart failure treatment. Furthermore, the data obtained show that β3-integrin expression is enhanced in human dilated and ischaemic cardiomyopathies.

Current treatments for heart failure are not disease modifying after treatment cessation and thus their efficacy relies on treatment for life (57). It is of interest that the effects of ldCil in the AAC model of heart failure show potentially long-lasting effects. The reasons why this vascular promotion effect inducing a pro-angiogenic transcriptomic profile may be effective compared with previous proangiogenic strategies such as protein tyrosine phosphatase 1B (PTP1B) inactivation (58, 59) or vascular endothelial growth factor-induced neovascularisation (60) are not known currently. One possibility is that endothelial cells that line blood vessels not only act as conduits for blood flow but also produce their own set of paracrine factors, namely angiocrine factors, that can have effects on development and the repair of surrounding tissue (61). This is a relatively new, but exciting, concept in understanding the role of cardiac angiocrine factors in cardiac repair (62). Recognising that angiocrine biology has been related to integrin mediated mechanosensing (63) or signalling downstream of endothelial cell-integrins (64) it is possible that angiocrine factor production after ldCil treatment may be of significance. Overall, it is likely that since ldCil treatment, as well as its proangiogenic effects, also has direct effects on cardiomyocyte function and hypertrophy, that the combination of these effects on at least 2 cell types in the heart provide improved restoration of the pathophysiological features of heart failure.

The in vitro data indicate that the effects of ldCil on AngII-stressed cardiomyocytes correlates with a transcriptomic signature of a published human normal heart compared with failing heart (52). The common transcriptomic signature suggests that if in vivo ldCil effects on cardiomyocytes are similar to those discovered in vitro, then ldCil treatment could have a restorative effect on cardiomyocyte pathophysiology that is relevant to human heart disease.

Overall, our results suggest that ldCil may be used as a potential preventative agent or treatment for heart failure with disease modifying effects, and may also have utility in preventing or treating vascular conditions.

### REFERENCES

1. Hajjar R J, Ishikawa K. Introducing Genes to the Heart: All About Delivery. Circ Res. 2017; 120(1):33-5.
2. Yla-Herttuala S, Bridges C, Katz M G, Korpisalo P. Angiogenic gene therapy in cardiovascular diseases: dream or vision? Eur Heart J. 2017; 38(18):1365-71.
3. Losordo D W, Vale P R, Symes J F, Dunnington C H, Esakof D D, Maysky M, et al. Gene therapy for myocardial angiogenesis: initial clinical results with direct myocardial injection of phVEGF165 as sole therapy for myocardial ischemia. Circulation. 1998; 98(25):2800-4.
4. Pearlman J D, Hibberd M G, Chuang M L, Harada K, Lopez J J, Gladstone S R, et al. Magnetic resonance mapping demonstrates benefits of VEGF-induced myocardial angiogenesis. Nat Med. 1995; 1(10):1085-9.
5. Hughes G C, Biswas S S, Yin B, Coleman R E, DeGrado T R, Landolfo C K, et al. Therapeutic angiogenesis in chronically ischemic porcine myocardium: comparative effects of bFGF and VEGF. Ann Thorac Surg. 2004; 77(3):812-8.
6. Zhou L, Ma W, Yang Z, Zhang F, Lu L, Ding Z, et al. VEGF165 and angiopoietin-1 decreased myocardium infarct size through phosphatidylinositol-3 kinase and Bcl-2 pathways. Gene Ther. 2005; 12(3):196-202.
7. Pepe M, Mamdani M, Zentilin L, Csiszar A, Qanud K, Zacchigna S, et al. Intramyocardial VEGF-B167 gene delivery delays the progression towards congestive failure in dogs with pacing-induced dilated cardiomyopathy. Circ Res. 2010; 106(12):1893-903.
8. Serpi R, Tolonen A M, Huusko J, Rysa J, Tenhunen O, Yla-Herttuala S, et al. Vascular endothelial growth factor-B gene transfer prevents angiotensin II-induced diastolic dysfunction via proliferation and capillary dilatation in rats. Cardiovasc Res. 2011; 89(1):204-13.
9. Huusko J, Lottonen L, Merentie M, Gurzeler E, Anisimov A, Miyanohara A, et al. AAV9-mediated VEGF-B gene transfer improves systolic function in progressive left ventricular hypertrophy. Mol Ther. 2012; 20(12):2212-21.
10. Battler A, Scheinowitz M, Bor A, Hasdai D, Vered Z, Di Segni E, et al. Intracoronary injection of basic fibroblast growth factor enhances angiogenesis in infarcted swine myocardium. J Am Coll Cardiol. 1993; 22(7):2001-6.
11. Laham R J, Chronos N A, Pike M, Leimbach M E, Udelson J E, Pearlman J D, et al. Intracoronary basic fibroblast growth factor (FGF-2) in patients with severe ischemic heart disease: results of a phase I open-label dose escalation study. J Am Coll Cardiol. 2000; 36(7):2132-9.
12. Hynes R O. Integrins: bidirectional, allosteric signaling machines. Cell. 2002; 110(6):673-87.
13. Ley K, Rivera-Nieves J, Sandborn W J, Shattil S. Integrin-based therapeutics: biological basis, clinical use and new drugs. Nat Rev Drug Discov. 2016; 15(3):173-83.
14. Robinson S D, Hodivala-Dilke K M. The role of beta3-integrins in tumor angiogenesis: context is everything. Curr Opin Cell Biol. 2011; 23(5):630-7.
15. Johnston R K, Balasubramanian S, Kasiganesan H, Baicu C F, Zile M R, Kuppuswamy D. Beta3 integrin-mediated ubiquitination activates survival signaling during myocardial hypertrophy. Faseb j. 2009; 23(8):2759-71.
16. Civitarese R A, Kapus A, McCulloch C A, Connelly K A. Role of integrins in mediating cardiac fibroblast-cardiomyocyte cross talk: a dynamic relationship in cardiac biology and pathophysiology. Basic Res Cardiol. 2017; 112(1):6.
17. Meoli D F, Sadeghi M M, Krassilnikova S, Bourke B N, Giordano F J, Dione D P, et al. Noninvasive imaging of myocardial angiogenesis following experimental myocardial infarction. J Clin Invest. 2004; 113(12):1684-91.
18. Sherif H M, Saraste A, Nekolla S G, Weidl E, Reder S, Tapfer A, et al. Molecular imaging of early alphavbeta3 integrin expression predicts long-term left-ventricle remodeling after myocardial infarction in rats. J Nucl Med. 2012; 53(2):318-23.
19. Gronman M, Tarkia M, Kiviniemi T, Halonen P, Kuivanen A, Savunen T, et al. Imaging of alphavbeta3 integrin expression in experimental myocardial ischemia with [(68)Ga]NODAGA-RGD positron emission tomography. J Transl Med. 2017; 15(1):144.
20. Jenkins W S A, Vesey A T, Stirrat C, Connell M, Lucatelli C, Neale A, et al. Cardiac αVβ3 integrin expression following acute myocardial infarction in humans. 2017.
21. Higuchi T, Bengel F M, Seidl S, Watzlowik P, Kessler H, Hegenloh R, et al. Assessment of alphavbeta3 integrin expression after myocardial infarction by positron emission tomography. Cardiovasc Res. 2008; 78(2):395-403.
22. Buerkle M A, Pahernik S A, Sutter A, Jonczyk A, Messmer K, Dellian M. Inhibition of the alpha-nu integrins with a cyclic RGD peptide impairs angiogenesis, growth and metastasis of solid tumours in vivo. Br J Cancer. 2002; 86(5):788-95.
23. Brooks P C, Clark R A, Cheresh D A. Requirement of vascular integrin alpha v beta 3 for angiogenesis. Science. 1994; 264(5158):569-71.
24. Nisato R E, Tille J C, Jonczyk A, Goodman S L, Pepper M S. alphav beta 3 and alphav beta 5 integrin antagonists inhibit angiogenesis in vitro. Angiogenesis. 2003; 6(2):105-19.
25. Maubant S, Saint-Dizier D, Boutillon M, Perron-Sierra F, Casara P J, Hickman J A, et al. Blockade of alpha v beta3 and alpha v beta5 integrins by RGD mimetics induces anoikis and not integrin-mediated death in human endothelial cells. Blood. 2006; 108(9):3035-44.
26. Nabors L B, Mikkelsen T, Rosenfeld S S, Hochberg F, Akella N S, Fisher J D, et al. Phase I and correlative biology study of cilengitide in patients with recurrent malignant glioma. J Clin Oncol. 2007; 25(13):1651-7.
27. Reynolds A R, Hart I R, Watson A R, Welti J C, Silva R G, Robinson S D, et al. Stimulation of tumor growth and angiogenesis by low concentrations of RGD-mimetic integrin inhibitors. Nat Med. 2009; 15(4):392-400.
28. Wong P P, Demircioglu F, Ghazaly E, Alrawashdeh W, Stratford M R, Scudamore C L, et al. Dual-action combination therapy enhances angiogenesis while reducing tumor growth and spread. Cancer Cell. 2015; 27(1):123-37.
29. deAlmeida A C, van Oort R J, Wehrens X H. Transverse aortic constriction in mice. J Vis Exp. 2010(38).
30. Songstad N T, Johansen D, How O J, Kaaresen P I, Ytrehus K, Acharya G. Effect of transverse aortic constriction on cardiac structure, function and gene expression in pregnant rats. PLoS One. 2014; 9(2):e89559.
31. Furihata T, Kinugawa S, Takada S, Fukushima A, Takahashi M, Homma T, et al. The experimental model of transition from compensated cardiac hypertrophy to failure created by transverse aortic constriction in mice. Int J Cardiol Heart Vasc. 2016; 11:24-8.
32. Bennis Y, Sarlon-Bartoli G, Guillet B, Lucas L, Pellegrini L, Velly L, et al. Priming of late endothelial progenitor cells with erythropoietin before transplantation requires the CD131 receptor subunit and enhances their angiogenic potential. J Thromb Haemost. 2012; 10(9):1914-28.
33. Hilgendorff A, Parai K, Ertsey R, Juliana Rey-Parra G, Thebaud B, Tamosiuniene R, et al. Neonatal mice genetically modified to express the elastase inhibitor elafin are protected against the adverse effects of mechanical ventilation on lung growth. Am J Physiol Lung Cell Mol Physiol. 2012; 303(3):L215-27.
34. Lin C G, Leu S J, Chen N, Tebeau C M, Lin S X, Yeung C Y, et al. CCN3 (NOV) is a novel angiogenic regulator of the CCN protein family. J Biol Chem. 2003; 278(26):24200-8.
35. Bousquenaud M, Schwartz C, Leonard F, Rolland-Turner M, Wagner D, Devaux Y. Monocyte chemotactic protein 3 is a homing factor for circulating angiogenic cells. Cardiovasc Res. 2012; 94(3):519-25.
36. Schuermann A, Helker C S, Herzog W. Metallothionein 2 regulates endothelial cell migration through transcriptional regulation of vegfc expression. Angiogenesis. 2015; 18(4):463-75.
37. Hoefer J, Kern J, Ofer P, Eder I E, Schafer G, Dietrich D, et al. SOCS2 correlates with malignancy and exerts growth-promoting effects in prostate cancer. Endocr Relat Cancer. 2014; 21(2):175-87.
38. Yanochko G M, Vitsky A, Heyen J R, Hirakawa B, Lam J L, May J, et al. Pan-FGFR inhibition leads to blockade of FGF23 signaling, soft tissue mineralization, and cardiovascular dysfunction. Toxicol Sci. 2013; 135(2):451-64.
39. Gurusamy K S, Farooqui N, Loizidou M, Dijk S, Taanman J W, Whiting S, et al. Influence of zinc and zinc chelator on HT-29 colorectal cell line. Biometals. 2011; 24(1):143-51.
40. Nanda A, Carson-Walter E B, Seaman S, Barber T D, Stampfl J, Singh S, et al. TEM8 interacts with the cleaved C5 domain of collagen alpha 3(VI). Cancer Res. 2004; 64(3):817-20.
41. Zhou Z, Apte S S, Soininen R, Cao R, Baaklini G Y, Rauser R W, et al. Impaired endochondral ossification and angiogenesis in mice deficient in membrane-type matrix metalloproteinase I. Proc Natl Acad Sci USA. 2000; 97(8):4052-7.
42. Moriwaki S, Into T, Suzuki K, Miyauchi M, Takata T, Shibayama K, et al. gamma-Glutamyltranspeptidase is an endogenous activator of Toll-like receptor 4-mediated osteoclastogenesis. Sci Rep. 2016; 6:35930.
43. Mahoney E T, Benton R L, Maddie M A, Whittemore S R, Hagg T. ADAM8 is selectively up-regulated in endothelial cells and is associated with angiogenesis after spinal cord injury in adult mice. J Comp Neurol. 2009; 512(2):243-55.
44. Liu Y, Leri A, Li B, Wang X, Cheng W, Kajstura J, et al. Angiotensin II stimulation in vitro induces hypertrophy of normal and postinfarcted ventricular myocytes. Circ Res. 1998; 82(11):1145-59.
45. Taegtmeyer H, Sen S, Vela D. Return to the fetal gene program: A suggested metabolic link to gene expression in the heart. Ann NY Acad Sci. 2010; 1188:191-8.
46. Arrington C B, Patel A, Bacino C A, Bowles N E. Haploinsufficiency of the LIM domain containing preferred translocation partner in lipoma (LPP) gene in patients with tetralogy of Fallot and VACTERL association. Am J Med Genet A. 2010; 152A(11):2919-23.
47. Liu Y H, Xu J, Yang X P, Yang F, Shesely E, Carretero O A. Effect of ACE inhibitors and angiotensin II type 1 receptor antagonists on endothelial NO synthase knockout mice with heart failure. Hypertension. 2002; 39(2 Pt 2):375-81.
48. Yadav S, Cotlarciuc I, Munroe P B, Khan M S, Nalls M A, Bevan S, et al. Genome-wide analysis of blood pressure variability and ischemic stroke. Stroke. 2013; 44(10):2703-9.
49. Santiago-Sim T, Burrage L C, Ebstein F, Tokita M J, Miller M, Bi W, et al. Biallelic Variants in OTUD6B Cause an Intellectual Disability Syndrome Associated with Seizures and Dysmorphic Features. Am J Hum Genet. 2017; 100(4):676-88.
50. Doenst T, Nguyen T D, Abel E D. Cardiac metabolism in heart failure: implications beyond ATP production. Circ Res. 2013; 113(6):709-24.
51. Aoyagi T, Matsui T. Phosphoinositide-3 kinase signaling in cardiac hypertrophy and heart failure. Curr Pharm Des. 2011; 17(18):1818-24.
52. Liu Y, Morley M, Brandimarto J, Hannenhalli S, Hu Y, Ashley E A, et al. RNA-Seq Identifies Novel Myocardial Gene Expression Signatures of Heart Failure. Genomics. 2015; 105(2):83-9.
53. Sun M, Opaysky M A, Stewart D J, Rabinovitch M, Dawood F, Wen W H, et al. Temporal response and localization of integrins beta1 and beta3 in the heart after myocardial infarction: regulation by cytokines. Circulation. 2003; 107(7):1046-52.
54. Dechantsreiter M A, Planker E, Matha B, Lohof E, Holzemann G, Jonczyk A, et al. N-Methylated cyclic RGD peptides as highly active and selective alpha(V)beta(3) integrin antagonists. J Med Chem. 1999; 42(16):3033-40.
55. Mas-Moruno C, Rechenmacher F, Kessler H. Cilengitide: the first anti-angiogenic small molecule drug candidate design, synthesis and clinical evaluation. Anticancer Agents Med Chem. 2010; 10(10):753-68.
56. Haubner R F, D. Kessler, H. Stereoisomeric Peptide Libraries and Peptidomimetics for Designing Selective Inhibitors of the avβ3 Integrin for a New Cancer Therapy. Angew Chem Int Ed Engl 1997; 109:1440-56.
57. Nabeebaccus A, Zheng S, Shah A M. Heart failure-potential new targets for therapy. Br Med Bull. 2016; 119(1):99-110.
58. Besnier M, Coquerel D, Favre J, Dumesnil A, Guerrot D, Remy-Jouet I, et al. Protein tyrosine phosphatase 1B inactivation limits aging-associated heart failure in mice. Am J Physiol Heart Circ Physiol. 2018; 314(6):H1279-H88.
59. Besnier M, Galaup A, Nicol L, Henry J P, Coquerel D, Gueret A, et al. Enhanced angiogenesis and increased cardiac perfusion after myocardial infarction in protein tyrosine phosphatase 1B-deficient mice. FASEB J. 2014; 28(8):3351-61.
60. Gordon O, Gilon D, He Z, May D, Lazarus A, Oppenheim A, et al. Vascular endothelial growth factor-induced neovascularization rescues cardiac function but not adverse remodeling at advanced ischemic heart disease. Arterioscler Thromb Vasc Biol. 2012; 32(7):1642-51.
61. Rafii S, Butler J M, Ding B S. Angiocrine functions of organ-specific endothelial cells. Nature. 2016; 529(7586):316-25.
62. Taylor J, Fischer A. Endothelial cells dictate cardiac fuel source. Aging (Albany N.Y.). 2019.
63. Lorenz L, Axnick J, Buschmann T, Henning C, Urner S, Fang S, et al. Mechanosensing by beta1 integrin induces angiocrine signals for liver growth and survival. Nature. 2018; 562(7725):128-32.
64. Tavora B, Reynolds L E, Batista S, Demircioglu F, Fernandez I, Lechertier T, et al. Endothelial—cell FAK targeting sensitizes tumours to DNA—damaging therapy. Nature. 2014; 514(7520):112-6.
65. Kim D, Langmead B, Salzberg S L. HISAT: a fast spliced aligner with low memory requirements. Nat Methods. 2015; 12(4):357-60.
66. Anders S, Pyl P T, Huber W. HTSeq—a Python framework to work with high-throughput sequencing data. Bioinformatics. 2015; 31(2):166-9.
67. Ritchie M E, Phipson B, Wu D, Hu Y, Law C W, Shi W, et al. limma powers differential expression analyses for RNA-sequencing and microarray studies. Nucleic Acids Res. 2015; 43(7):e47.
68. Subramanian A, Tamayo P, Mootha V K, Mukherjee S, Ebert B L, Gillette M A, et al. Gene set enrichment analysis: a knowledge-based approach for interpreting genome-wide expression profiles. Proc Natl Acad Sci USA. 2005; 102(43):15545-50.
69. G. Müller, M. Gurrath, M. Kurz, H. Kessler; βVI Turns in Peptides and Proteins: A Model Peptide Mimicry; Proteins: Structure, Function, and Genetics 1993, 15, 235-251.
70. A. Schumacher-Klinger, J. Fanous, S. Merzbach, M. Weinmueller, F. Reichart, A. Rader, A. Domaglaska, C. Gilon, H. Kessler, A. Hoffman, Enhancing oral bioavailability of cyclic RGD hexa-peptides by the Lipophilic Prodrug Charge Masking approach: Redirection of peptide intestinal permeability from paracellular to transcellular pathway, Molecular Pharmaceutics 2018, 15. 3468-3477.

1. A method of preventing or treating a vascular condition or heart failure in a patient, comprising administering an αvβ3- and/or αvβ5-integrin targeting agent to the patient.

2. The method of claim 1, wherein said heart failure comprises hypertrophic, dilated or ischaemic cardiomyopathy, and/or coronary microvascular disease.

3. The method of claim 1, wherein said vascular condition is cardiovascular disease, peripheral vascular disease, or ischemia.

4. The method of any one of claims 1-3, wherein the αvβ3- and/or αvβ5-integrin targeting agent is a small molecule, a peptide, a peptidomimetic, a protein, or an antibody

5. The method of claim 4, wherein the αvβ3- and/or αvβ5-integrin targeting agent is a peptide comprising an RGD motif or a peptidomimetic thereof.

6. The method of claim 5, wherein the peptide or peptidomimetic is a cyclic peptide of about five to about 8 amino acids in length or a peptidomimetic thereof.

7. The method of claim 6 wherein the peptide or peptidomimetic is a pentapeptide or a hexapeptide or a peptidomimetic thereof.

8. The method of any one of claims 6 to 7, wherein the peptide or peptidomimetic is a said peptide comprising at least one N-alkylated amino acid residue, optionally at least one N-methylated amino acid residue, or a peptidomimetic thereof.

9. The method of any one of claims 5 to 8, wherein the peptide or peptidomimetic is a said peptide comprising at least one D-amino acid or Gly, or a peptidomimetic thereof, preferably wherein said peptide or peptidomimetic comprises at least one βII′ turn and the at least one D-amino acid or Gly is provided at the i+1 position of the βII′ turn.

10. The method of any one of claims 5 to 9, wherein the peptide or peptidomimetic is a said peptide comprising at least one hydrophobic amino acid residue, optionally selected from Val or Phe, or a peptidomimetic thereof

11. The method of any one of claims 5 to 10, wherein the peptide is selected from cilengitide or a derivative or analogue or peptidomimetic thereof, or one of the following peptides or a derivative or analogue or peptidomimetic thereof:
(SEQ ID NO: 1)
a. *rGDA*AA;
(SEQ ID NO: 13)
b. *rGDAA*A;
(SEQ ID NO: 2)
c. *aRGDA*A;
(SEQ ID NO: 3)
d. rG*DA*AA;
(SEQ ID NO: 4)
e. rGDA*A*A;
(SEQ ID NO: 5)
f. *vRGDA*A;
(SEQ ID NO: 6)
g. *fRGDA*A;
(SEQ ID NO: 7)
h. *rGDA*AV;
and
(SEQ ID NO: 8)
i. *rGDA*AF;
wherein * represents N-methylation of the following amino acid, R is arginine, G is glycine, D is aspartic acid, r is arginine in the D configuration, A is alanine, a is alanine in the D configuration, V is valine, v is valine in the D configuration, F is phenylalanine, and f is phenylalanine in the D configuration, and preferably the peptide is a cyclic peptide of any one of SEQ ID NOs 24-31 and 36, or a derivative or analogue or peptidomimetic thereof.

12. The method of claim 11, wherein the peptide is *vRGDA*A (SEQ ID NO: 5), a cyclic peptide of SEQ ID NO: 28, or a derivative or analogue or peptidomimetic of either thereof.

13. The method of any one of claims 4 to 12, wherein the peptide or peptidomimetic is in the form of a prodrug, or is provided as a conjugate or fusion protein.

14. The method of claim 13, wherein the prodrug comprises at least one moiety that reduces net charge of the peptide or peptidomimetic, and/or comprises at least one of the following moieties or groups of moieties:
(i) a —CO₂R moiety, wherein R is alkyl;
(ii) a hexyloxycarbonyl (Hoc) moiety, optionally linked to an arginine residue;
(iii) a methyl ester moiety (OMe)or other alkyl ester moiety, such as a methyl or alkyl ester of Asp;
(iv) hexyloxycarbonyl (Hoc) and ester (OMe) moieties; and/or
(v) two hexyloxycarbonyl (Hoc) moieties;
optionally wherein the prodrug has the formula: c(*vR(Hoc)₂GD(OMe)A*A)(SEQ ID NO: 9), c(*aR(Hoc)₂GD(OMe)A*A)(SEQ ID NO: 10), c(*r(Hoc)₂GD(OMe)A*AA)(SEQ ID NO: 11) or c(r(Hoc)₂GD(OMe)A*A*A)(SEQ ID NO: 12).

15. The method of any one of claims 4 to 14, wherein the small molecule, peptide or peptidomimetic is administered orally.

16. The method of any one of claims 4 to 15, wherein the peptide or peptidomimetic is administered as a pharmaceutical composition comprising said peptide or peptidomimetic, a pharmaceutically acceptable carrier, excipient or diluent.

17. The method of claim 16, wherein said composition comprises at least one absorption enhancer.

18. An αvβ3- and/or αvβ5-integrin targeting agent for use in a method of preventing or treating a vascular condition or heart failure in a patient.

19. A combination of an αvβ3- and/or αvβ5-integrin targeting agent and at least one additional agent suitable for preventing or treating a vascular condition, heart failure or symptoms of heart failure.

20. The combination according to claim 19, wherein said αvβ3- and/or αvβ5-integrin targeting agent is a peptide or peptidomimetic as defined in any one of claims 4 to 14.

21. The combination according to claim 19 or 20, which is suitable for oral administration.
